SEQUENCE LISTING (27 sequences)

SEQ ID NO: 1 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 2 - protein, 6 aa, Artificial Sequence
  VCAM-1 signaling peptide
QIDSPL

SEQ ID NO: 3 - protein, 8 aa, Artificial Sequence
  VCAM-1 signaling peptide
TQIDSPLN

SEQ ID NO: 4 - protein, 739 aa, Mus musculus
MPVKMVAVLGASTVLWILFAVSQAFKIEISPEYKTIAQIGDSMALTCSTTGCESPLFSWRTQIDSPLNAKVRTEGSKSVLTMEPVSFENEHSYLCTATCGSGKLERSIHVDIYSFPKDPEIQFSGPLEVGKPVTVKCLAPDIYPVYRLEIDLFKGDQLMNRQEFSSEEMTKSLETKSLEVTFTPVIEDIGKALVCRAKLHIDQIDSTLKERETVKELQVYISPRNTTISVHPSTRLQEGGAVTMTCSSEGLPAPEIFWGRKLDNEVLQLLSGNATLTLIAMRMEDSGVYVCEGVNLIGRDKAEVELVVQEKPFIVDISPGSQVAAQVGDSVVLTCAAIGCDSPSFSWRTQTDSPLNGVVRNEGAKSTLVLSSVGFEDEHSYLCAVTCLQRTLEKRTQVEVYSFPEDPVIKMSGPLVHGRPVTVNCTVPNVYPFDHLEIELLKGETTLMKKYFLEEMGIKSLETKILETTFIPTIEDTGKSLVCLARLHSGEMESEPKQRQSVQPLYVNVAPKETTIWVSPSPILEEGSPVNLTCSSDGIPAPKILWSRQLNNGELQPLSENTTLTFMSTKRDDSGIYVCEGINEAGISRKSVELIIQVSPKDIQLTVFPSKSVKEGDTVIISCTCGNVPETWIILKKKAKTGDMVLKSVDGSYTIRQAQLQDAGIYECESKTEVGSQLRSLTLDVKGKEHNKDYFSPELLALYCASSLVIPAIGMIVYFARKANMKGSYSLVEAQKSKV

SEQ ID NO: 5 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 6 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 7 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 8 - DNA, 21 bp, Artificial Sequence
  Human ACTB forward primer
catgtacgttgctatccaggc

SEQ ID NO: 9 - DNA, 21 bp, Artificial Sequence
  Human ACTB reverse primer
ctccttaatgtcacgcacgat

SEQ ID NO: 10 - DNA, 21 bp, Artificial Sequence
  Human BCL11B forward primer
tccagctacatttgcacaaca

SEQ ID NO: 11 - DNA, 20 bp, Artificial Sequence
  Human BCL11B reverse primer
gctccaggtagatgcggaag

SEQ ID NO: 12 - DNA, 20 bp, Artificial Sequence
  Human DTX1 forward primer
atcggagaaggctctacagg

SEQ ID NO: 13 - DNA, 21 bp, Artificial Sequence
  Human DTX1 reverse primer
cgtctggcctcctttctaact

SEQ ID NO: 14 - DNA, 20 bp, Artificial Sequence
  Human E2A forward primer
ccgactcctacagtgggcta

SEQ ID NO: 15 - DNA, 19 bp, Artificial Sequence
  Human E2A reverse primer
cgctgacgtgttctcctcg

SEQ ID NO: 16 - DNA, 20 bp, Artificial Sequence
  Human GATA3 forward primer
gttggcctaaggtggttgtg

SEQ ID NO: 17 - DNA, 20 bp, Artificial Sequence
  Human GATA3 reverse primer
acaggctgcaggaataggga

SEQ ID NO: 18 - DNA, 20 bp, Artificial Sequence
  Human HES1 forward primer
cctgtcatccccgtctacac

SEQ ID NO: 19 - DNA, 19 bp, Artificial Sequence
  Human HES1 reverse primer
cacatggagtccgccgtaa

SEQ ID NO: 20 - DNA, 19 bp, Artificial Sequence
  Human NOTCH1 forward primer
gaggcgtggcagactatgc

SEQ ID NO: 21 - DNA, 20 bp, Artificial Sequence
  Human NOTCH1 reverse primer
cttgtactccgtcagcgtga

SEQ ID NO: 22 - DNA, 20 bp, Artificial Sequence
  Human SPI1 forward primer
tgcaatgtcaagggaggggg

SEQ ID NO: 23 - DNA, 21 bp, Artificial Sequence
  Human SPI1 reverse primer
aaacccttccattttgcacgc

SEQ ID NO: 24 - DNA, 21 bp, Artificial Sequence
  Human TCF7 forward primer
tgcacatgcagctatacccag

SEQ ID NO: 25 - DNA, 22 bp, Artificial Sequence
  Human TCF7 reverse primer
tggtggattcttggtgcttttc

SEQ ID NO: 26 - DNA, 18 bp, Artificial Sequence
  Human CEBPA forward primer
ggagctgagatcccgaca

SEQ ID NO: 27 - DNA, 20 bp, Artificial Sequence
  Human CEBPA reverse primer
ttctaaggacaggcgtggag

(19) United States
(12) Patent Application Publication
(10) Pub. No.: US 2022/0403326 A1
(43) Pub. Date: Dec. 22, 2022

(54) MEDIA FORMULATIONS AND METHODS FOR PRODUCING PROGENITOR T CELLS

(71) Applicants: Peter William ZANDSTRA, Vancouver (CA); John Michael EDGAR, Vancouver (CA)

(72) Inventors: Peter William ZANDSTRA, Vancouver (CA); John Michael EDGAR, Vancouver (CA)

(21) Appl. No.: 17/776,575

(22) PCT Filed: Nov. 13, 2020
(86) PCT No.: PCT/CA2020/051554

§ 371 Date: May 12, 2022

Related U.S. Application Data
(60) Provisional application No. 62/935,296, filed on Nov. 14, 2019.

(51) Int. Cl. C12N 5/00; C12N 5/0783; A61K 35/17

(52) U.S. Cl. CPC C12N 5/0037; C12N 5/0636; C12N 5/0646; A61K 35/17; C12N 2501/25; C12N 2501/2303; C12N 2501/42; C12N 2501/26; C12N 2501/125; C12N 2501/2307; C12N 2501/585; C12N 2501/145; C12N 2506/11; C12N 2500/25; C12N 2500/90; C12N 2500/36; C12N 2500/38

(57) ABSTRACT

The present disclosure relates generally to culture media formulations and culture methods. More particularly, the present disclosure provides defined serum-free culture media, kits and methods for generating progenitor T cells and derivatives thereof, including mature T cells. The present disclosure further provides the cells generated using the media, kits and methods, as well as methods of treatment using the generated cells.

### CROSS REFERENCE TO PRIOR APPLICATIONS

This application claims priority under the Paris Convention to U.S. Provisional Patent Application 62/935,296, filed Nov. 14, 2019, which is incorporated herein by reference as if set forth in its entirety.

### FIELD OF THE DISCLOSURE

The present disclosure relates generally to culture media formulations and culture methods. More particularly, the present disclosure relates to defined culture media and in vitro methods for generating progenitor T (proT) cells and derivatives of proT cells from stem and/or progenitor cells.

### BACKGROUND OF THE DISCLOSURE

T cells are a type of lymphocyte that play a central role in cell-mediated immunity. For example, T cells are involved with regulating immune responses and maintaining an immunological memory of recurring pathogens in the body. T cell deficiency can be lethal, particularly in post-chemotherapy patients, who are at an increased risk for opportunistic infections.

Conventional in vitro T cell development from hematopoietic stem and progenitor cells (HSPCs) is carried out in serum-containing medium and on murine OP9 feeders engineered to express Notch-activating Delta-like 4 (DL4) protein.^{1,2} The undefined and xenogeneic nature of this system makes it difficult to study the role of endogenously secreted factors or extracellular matrix components, and limits its clinical translation. It has been reported that use of an OP9 feeder layer can be avoided by non-specific adsorption of Notch ligands to tissue culture plates.³ However, this OP9-free system required use of high amounts of animal sera in the culture medium. Immobilization of DL4 to magnetic microbeads has also been reported as an artificial Notch signaling system. However, this approach suffered from skewing to non-T lineage cells, such as B lineage cells.⁴ In one study, Notch ligand Jagged1-Fc was robotically spotted on microfabricated pillars, stamped on thin thiolated PEG hydrogel films and tethered via maleimide-modified Protein A to study its effects on self-renewal of single neural stem cells.⁵ However, there is no evidence to suggest that this small scale, single cell approach would be suitable for translation to T cell development for clinical applications.

There is a need in the art for improved formulations and methods to facilitate clinical translation of T cell-based immunotherapies.

### SUMMARY OF THE DISCLOSURE

The inventors have invented defined cell culture media formulations, kits and in vitro methods for generating proT cells and derivatives of proT cells, including mature T cells, and in vitro methods for generating natural killer (NK) cells and derivatives of NK cells from stem and/or progenitor cells.

In a first aspect of the disclosure, a serum-free culture medium is provided. The serum-free culture medium comprises tumor necrosis factor alpha (TNFα), interleukin-3 (IL-3) and a Notch ligand, or functionally equivalent fragments thereof, wherein the Notch ligand or fragment thereof is adsorbed or immobilized to a substrate.

In an embodiment of the serum-free culture medium provided herein, the medium further comprises at least one of: stem cell factor (SCF), interleukin 7 (IL-7) or FMS-like tyrosine kinase 3 ligand (FLT3L), or functionally equivalent fragments thereof. In an embodiment of the serum-free culture medium provided herein, the medium further comprises at least two of: stem cell factor (SCF), interleukin 7 (IL-7) or FMS-like tyrosine kinase 3 ligand (FLT3L), or functionally equivalent fragments thereof. In an embodiment of the serum-free culture medium provided herein, the medium further comprises stem cell factor (SCF), interleukin 7 (IL-7) and FMS-like tyrosine kinase 3 ligand (FLT3L) or functionally equivalent fragments thereof.

In an embodiment of the serum-free culture medium provided herein, the medium further comprises an adhesion ligand and/or an integrin ligand, or a functionally equivalent fragment thereof, adsorbed or immobilized to a substrate.

In an embodiment of the serum-free culture medium provided herein, the medium further comprises thrombopoietin (TPO) or a functionally equivalent fragment thereof.

In an embodiment of the serum-free culture medium provided herein, the TNFα is present at a concentration of between about 1 ng/ml and about 50 ng/ml.

In an embodiment of the serum-free culture medium provided herein, the IL-3 is present at a concentration of between about 1 ng/ml and about 50 ng/ml.

In an embodiment of the serum-free culture medium provided herein, the SCF, IL-7 and FLT3L, when present, are each present at a concentration of between about 5 ng/ml and about 200 ng/ml.

In an embodiment of the serum-free culture medium provided herein, the Notch ligand is Delta-like 4 (DL4), Delta-like 1 (DL1), Jagged 2 (JAG2), or a combination thereof.

In an embodiment of the serum-free culture medium provided herein, the substrate comprises one or more beads contained within the medium.

In an embodiment of the serum-free culture medium provided herein, the Notch ligand is adsorbed or immobilized to the substrate at a coating concentration of between about 0.5 ng/mm² and about 50 ng/mm².

In an embodiment of the serum-free culture medium provided herein, the medium further comprises one or more of bovine serum albumin, insulin, transferrin, low-density lipoprotein, ascorbic acid, 2-mercaptoethanol, penicillin and streptomycin.

In a second aspect of the disclosure, a kit is provided. The kit comprises the serum free culture medium of the first aspect and at least one container.

In a third aspect of the disclosure, a kit is provided. The kit comprises: (a) a serum-free culture medium comprising TNFα, IL-3, or functionally equivalent fragments thereof; and (b) a coating medium comprising a Notch ligand or a functionally equivalent fragment thereof.

In an embodiment of the kit comprising a serum-free culture medium and a coating medium provided herein, the serum-free culture medium further comprises at least one of: stem cell factor (SCF), interleukin 7 (IL-7) or FMS-like tyrosine kinase 3 ligand (FLT3L), or functionally equivalent fragments thereof. In an embodiment of the kit comprising a serum-free culture medium and a coating medium provided herein, the serum-free culture medium further comprises at least two of: stem cell factor (SCF), interleukin 7 (IL-7) or FMS-like tyrosine kinase 3 ligand (FLT3L), or functionally equivalent fragments thereof. In an embodiment of the kit comprising a serum-free culture medium and a coating medium provided herein, the serum-free medium further comprises stem cell factor (SCF), interleukin 7 (IL-7) and FMS-like tyrosine kinase 3 ligand (FLT3L), or functionally equivalent fragments thereof.

In an embodiment of the kit comprising a serum-free culture medium and a coating medium provided herein, the serum-free culture medium further comprises TPO or a functionally equivalent fragment thereof.

In an embodiment of the kit comprising a serum-free culture medium and a coating medium provided herein, the coating medium further comprises an adhesion ligand and/or an integrin ligand, or a functionally equivalent fragment thereof.

In an embodiment of the kit comprising a serum-free culture medium and a coating medium provided herein, the Notch ligand is DL4, DL1, JAG2, or a combination thereof.

In an embodiment of the kit comprising a serum-free culture medium and a coating medium provided herein, the kit further comprises one or more cell culture plates or dishes.

In a fourth aspect of the disclosure, a method of generating a proT cell or a derivative thereof is provided. The method comprises culturing stem and/or progenitor cells in a serum-free medium in the presence of TNFα, IL-3 and a Notch ligand or functionally equivalent fragments thereof, wherein the Notch ligand is adsorbed or immobilized to a substrate.

In an embodiment of the method of generating proT cells provided herein, the derivative is a preT cell, immature T cell or mature T cell.

In an embodiment of the method of generating proT cells provided herein, the method further comprises differentiating the progenitor T cell to a preT cell, immature T cell or a mature T cell.

In a fifth aspect of the disclosure, a method of generating natural killer (NK) cells or derivatives thereof is provided. The method comprises culturing stem and/or progenitor cells in a serum-free medium in the presence of TNFα, IL-3 and a Notch ligand or functionally equivalent fragments thereof, wherein the Notch ligand is adsorbed or immobilized to a substrate.

In an embodiment of the method of generating proT cells or NK cells provided herein, the culturing is further in the presence of at least one of: SCF, IL-7 or FLT3L, or functionally equivalent fragments thereof. In an embodiment of the method of generating proT cells or NK cells provided herein, the culturing is further in the presence of at least two of: SCF, IL-7 or FLT3L, or functionally equivalent fragments thereof. In an embodiment of the method of generating proT cells or NK cells provided herein, the culturing is further in the presence of SCF, IL-7 and FLT3L, or functionally equivalent fragments thereof.

In an embodiment of the method of generating proT cells or NK cells provided herein, the TNFα and the IL-3 are provided to the cells at the same time.

In an embodiment of the method of generating proT cells or NK cells provided herein, the TNFα is provided to the cells prior to the IL-3.

In an embodiment of the method of generating proT cells or NK cells provided herein, the TNFα is provided to the cells 1, 2, 3, 4 or 5 days prior to the IL-3.

In an embodiment of the method of generating proT cells or NK cells provided herein, the culturing is further in the presence of an adhesion ligand and/or an integrin ligand, or a functionally equivalent fragment thereof, adsorbed or immobilized to a substrate.

In an embodiment of the method of generating proT cells or NK cells provided herein, the culturing is further in the presence of TPO or a functionally equivalent fragment thereof.

In an embodiment of the method of generating proT cells or NK cells provided herein, the TNFα is provided at a concentration of between about 1 ng/ml and about 50 ng/ml in the culture.

In an embodiment of the method of generating proT cells or NK cells provided herein, the IL-3 is provided at a concentration of between about 1 ng/ml and about 50 ng/ml in the culture.

In an embodiment of the method of generating proT cells or NK cells provided herein, the SCF, IL-7 and FLT3L are each provided at a concentration of between about 5 ng/ml and about 200 ng/ml in the culture.

In an embodiment of the method of generating proT cells or NK cells provided herein, the Notch ligand is DL4, DL1, JAG2, or a combination thereof.

In an embodiment of the method of generating proT cells or NK cells provided herein, the Notch ligand is adsorbed or immobilized to the substrate at a coating concentration of between about 0.5 ng/mm² and about 50 ng/mm².

In an embodiment of the method of generating proT cells or NK cells provided herein, the substrate comprises a surface of a cell culture plate or dish, or a culture bag.

In an embodiment of the method of generating proT cells or NK cells provided herein, the substrate comprises one or more beads contained within the medium.

In an embodiment of the method of generating proT cells or NK cells provided herein, the stem and/or progenitor cells are human cells.

In an embodiment of the method of generating proT cells or NK cells provided herein, the stem and/or progenitor cells are CD34+ hematopoietic stem and progenitor cells (HSPCs).

In an embodiment of the method of generating proT cells or NK cells provided herein, the CD34+ HSPCs are derived from umbilical cord blood, peripheral blood, bone marrow, embryonic stem cells or induced pluripotent stem cells.

In an embodiment of the method of generating proT cells provided herein, the proT cells comprise CD7+ cells, CD7+CD5+ cells, CD7+CD5+CD34+, CD7+CD5+CD45RA+ cell and/or CD7+CD5+CD1a+ cell.

In an embodiment of the method of generating NK cells provided herein, the NK cells comprise CD7+CD56+ cells.

In an embodiment of the method of generating proT cells provided herein, the generated proT cells have a higher cell surface density of IL-3 receptors compared to proT cells generated by culturing in a medium without TNFα.

In an embodiment of the method of generating proT cells provided herein, the culturing further comprises generating proT cells and, optionally, derivatives of the generated proT cells.

In an embodiment of the method of generating proT cells provided herein, the derivatives of the generated proT cells comprise CD4+CD8+ cells, CD4+CD3+ cells and/or CD8+CD3+ cells.

In an embodiment of the method of generating NK cells provided herein, the culturing further comprises generating NK cells and, optionally, derivatives of the generated NK cells.

In an embodiment of the method of generating proT cells or NK cells provided herein, the culturing is for a time of at least 7 days or at least 14 days.

In a sixth aspect of the disclosure, a proT cell or a derivative of a proT cell generated by the method of the fourth aspect is provided.

In a seventh aspect of the disclosure, a population of progenitor T cells made by the method according the fourth aspect is provided.

In an eighth aspect of the disclosure, an NK cell or a derivative of an NK cell generated by the method of the fifth aspect is provided.

In a ninth aspect of the disclosure, a population of NK cells made by the method according the fifth aspect is provided.

In a tenth aspect of the disclosure, a method for enhancing the immune response in a subject is provided. The method comprises administering to the subject an effective number of the proT cells or derivatives thereof of the sixth aspect.

In an eleventh aspect of the disclosure, a method for increasing the number of T cells in a subject is provided. The method comprises administering to the subject an effective number of the proT cells or derivatives thereof of the sixth aspect.

In a twelfth aspect of the disclosure, a method for enhancing the immune response in a subject is provided. The method comprises administering to the subject an effective number of the NK cells or derivatives thereof of the eighth aspect.

In a thirteenth aspect of the disclosure, a method for increasing the number of NK cells in a subject is provided. The method comprises administering to the subject an effective number of the NK cells or derivatives thereof of the eighth aspect.

In various embodiments of the method for enhancing the immune response in a subject or the method for increasing the number of T cells in a subject provided herein, the administered proT cells or derivatives thereof are autologous.

In various embodiments of the method for enhancing the immune response in a subject or the method for increasing the number of T cells in a subject provided herein, the administered proT cells or derivatives thereof are allogeneic.

In various embodiments of the method for enhancing the immune response in a subject or the method for increasing the number of NK cells in a subject provided herein, the administered NK cells or derivatives thereof are autologous.

In various embodiments of the method for enhancing the immune response in a subject or the method for increasing the number of NK cells in a subject provided herein, the administered NK cells or derivatives thereof are allogeneic.

In various embodiments of the method for enhancing the immune response in a subject or the method for increasing the number of T cells or NK cells in a subject provided herein, the subject has or is at risk of having an immune deficiency.

In various embodiments of the method for enhancing the immune response in a subject or the method for increasing the number of T cells in a subject provided herein, the immune deficiency is a T cell deficiency.

In various embodiments of the method for enhancing the immune response in a subject or the method for increasing the number of NK cells in a subject provided herein, the immune deficiency is an NK cell deficiency.

In various embodiments of the method for enhancing the immune response in a subject or the method for increasing the number of T cells or NK cells in a subject provided herein, the immune deficiency is caused by a medical condition, a chemical exposure and/or a radiation exposure.

In various embodiments of the method for enhancing the immune response in a subject or the method for increasing the number of T cells or NK cells in a subject provided herein, the medical condition is a cancer, a bone marrow failure, an anemia, a primary immunodeficiency disorder, an autoimmune disease, a partial thymectomy, an organ transplant, a viral infection, a bacterial infection, a fungal infection and/or idiopathic CD4+ T-lymphocytopenia.

In various embodiments of the method for enhancing the immune response in a subject or the method for increasing the number of T cells or NK cells in a subject provided herein, the viral infection is a human immunodeficiency virus (HIV) infection.

In various embodiments of the method for enhancing the immune response in a subject or the method for increasing the number of T cells or NK cells in a subject provided herein, the chemical exposure comprises chemotherapy, anti-inflammatory drug therapy, workplace-related chemical exposure or accidental poisoning with a chemical substance.

In various embodiments of the method for enhancing the immune response in a subject or the method for increasing the number of T cells or NK cells in a subject provided herein, the radiation exposure comprises radiotherapy, workplace-related radioisotope exposure, accidental poisoning with a radioisotope, nuclear meltdown or nuclear fallout.

In various embodiments of the method for enhancing the immune response in a subject or the method for increasing the number of T cells or NK cells in a subject provided herein, the subject is a human.

In a fourteenth aspect of the disclosure, a method of enhancing an immune response in a patient in need thereof by producing a population of progenitor T cells or derivatives thereof according to the method of the fourth aspect and administering the population to the patient.

In a fifteenth aspect of the disclosure, a method of enhancing an immune response in a patient in need thereof by producing a population of NK cells or derivatives thereof according to the method of the fifth aspect and administering the population to the patient.

In various embodiments of the method for enhancing the immune response in a patient in need thereof provided herein, the administered proT cells or derivatives thereof are autologous.

In various embodiments of the method for enhancing the immune response in a patient in need thereof provided herein, the administered proT cells or derivatives thereof are allogeneic.

In various embodiments of the method for enhancing the immune response in a patient in need thereof provided herein, the administered NK cells or derivatives thereof are autologous.

In various embodiments of the method for enhancing the immune response in a patient in need thereof provided herein, the administered NK cells or derivatives thereof are allogeneic.

In various embodiments of the method for enhancing the immune response in a patient in need thereof provided herein, the subject has or is at risk of having an immune deficiency.

In various embodiments of the method for enhancing the immune response in a patient in need thereof provided herein, the immune deficiency is a T cell deficiency.

In various embodiments of the method for enhancing the immune response in a patient in need thereof provided herein, the immune deficiency is an NK cell deficiency.

In various embodiments of the method for enhancing the immune response in a patient in need thereof provided herein, the immune deficiency is caused by a medical condition, a chemical exposure and/or a radiation exposure.

In various embodiments of the method for enhancing the immune response in a patient in need thereof provided herein, the medical condition is a cancer, a bone marrow failure, an anemia, a primary immunodeficiency disorder, an autoimmune disease, a partial thymectomy, an organ transplant, a viral infection, a bacterial infection, a fungal infection and/or idiopathic CD4+ T-lymphocytopenia.

In various embodiments of the method for enhancing the immune response in a patient in need thereof provided herein, the viral infection is a human immunodeficiency virus (HIV) infection.

In various embodiments of the method for enhancing the immune response in a patient in need thereof provided herein, the chemical exposure comprises chemotherapy, anti-inflammatory drug therapy, workplace-related chemical exposure or accidental poisoning with a chemical substance.

In various embodiments of the method for enhancing the immune response in a patient in need thereof provided herein, the radiation exposure comprises radiotherapy, workplace-related radioisotope exposure, accidental poisoning with a radioisotope, nuclear meltdown or nuclear fallout.

In various embodiments of the method for enhancing the immune response in a patient in need thereof provided herein, the subject is a human.

### BRIEF DESCRIPTION OF THE DRAWINGS

In order that the subject matter may be readily understood, embodiments are illustrated by way of non-limiting examples in the accompanying drawings.

FIG. 1a is a schematic of the experimental design of a screen for identifying new factors that enhance proT cell differentiation and expansion.

FIG. 1B is a set of graphs showing the expansion of total cells, CD7+CD5+ proT cells, CD7+CD56+ natural killer (NK) cells, and CD14/33+ myeloid cells at day 7 and day 14 in the presence of SCF, FLT3L, TPO and IL-7 (“4F cytokines”) at a concentration of 100 ng/ml or 20 ng/ml with respect to each cytokine. *p<0.05 from n=6 independent umbilical cord blood (UCB) donors; ns=not significant.

FIG. 1c is a plot showing the results of a screen to identify factors that enhance proT cell differentiation and expansion. The effect of each screened factor on total live cells, CD7=lymphocytes, and CD7+CD5+ proT cells is shown. Medium gray indicates the effect of the control condition; darker shades of gray indicate effects greater than the control condition; and lighter shades of gray indicate effects lesser than the control condition. The size of each circle indicates the significance of the effect in the linear regression model. Combinations of factors indicate two-factor interactions (e.g., TPO*IL-1β) or quadratic effects (e.g., IL-21*IL-21) in the regression model. n=2 independent UCB donors.

FIG. 1d is a set of graphs showing the dose response of live cells, CD7+ lymphocytes, and CD7+CD5+ proT cells to IL-3 and TNFα at the indicated range of concentrations. n=2 independent UCB donors.

FIG. 2a is a set of histograms of carboxyfluorescein succinimidyl ester (CFSE) stained cells showing the number of divisions of each cell through days 2 to 5 in the presence or absence of treatment with IL-3 or TNFα.

FIG. 2b is a set of graphs showing proliferation statistics from CFSE data of FIG. 2a and the frequency of CD7+ cells on each day. *p<0.05 relative to the control on each day. Results are shown as mean±standard error from n=4 independent UCB donors.

FIG. 2c is a set of flow cytometry plots showing the frequency of proT1 (CD34+CD7+CD5−) and proT2 (CD34+CD7+CD5+) cells on day 5 of the experiment shown in FIGS. 2a and b. The proT1 and proT2 cell populations were as defined by Awong et al.³

FIG. 3a is a schematic illustrating the involvement of TNFα in the NF-KB signalling pathway, which may regulate the Notch signalling pathway by regulating Notch target genes or Notch itself.

FIG. 3b is a flow cytometry plot and a schematic of the experimental design of a study investigating if and how TNFα interacts with the Notch pathway. Gene expression was measured using quantitative real-time PCR (qRT-PCR) after 5 days of culture.

FIG. 3c is a set of graphs showing the expression of the indicated Notch target genes (normalized to β-actin expression) in the presence or absence of TNFα. *p<0.05 from n=4 independent UCB donors.

FIG. 3d is a set of graphs showing the expression of the indicated pro-myeloid genes (normalized to β-actin expression) in the presence or absence of TNFα. *p<0.05 from n=4 independent UCB donors.

FIG. 3e is a schematic of the experimental design to elucidate stage-specific effects of TNFα on T lineage development.

FIG. 3f is a set of graphs showing the total number of cells and frequency of CD7+ and CD14/33+ cells on day 7 of the experiment shown in FIG. 3e. *p<0.05 relative to TNFα addition at day 0 for the frequency of CD7+ and CD14/33+ cells. Results are shown as mean±standard error from n=3 independent UCB donors; n.s.=not significant.

FIG. 4a is a schematic of a titration experiment to determine the effects of TNFα on proT cell development in the presence of an increasing concentration of an inhibitor of Notch activation, γ-secretase inhibitor (GSI).

FIG. 4b is a set of graphs showing the frequency and log 2-fold change of CD7+CD5+ cells in the presence of increasing concentrations of GSI. *p<0.05 relative to 0 μM GSI for the control and +TNFα conditions.

FIG. 4c is a set of representative flow cytometry plots showing the differential effects of GSI-mediated Notch inhibition on proT cell induction in the presence or absence of TNFα.

FIG. 4d is a schematic of a titration experiment to determine the effects of TNFα on proT cell development in the presence of a decreasing concentration of DL4.

FIG. 4e is a set of graphs showing the frequency and log 2-fold change of CD7+CD5+ cells in the presence of decreasing concentrations of DL4. *p<0.05 relative to 12 ng/mm² DL4 for the control and +TNFα conditions.

FIG. 4f is a set of representative flow cytometry plots showing the differential effects of DL4-mediated Notch activation on proT cell induction in the presence or absence of TNFα. Results are shown as mean±standard error from n=7 independent UCB donors.

FIG. 5a is a schematic of an experiment to determine the effects of combining IL-3 with TNFα on T lineage development. CD34+ HSPCs were placed on DL4+VCAM-1 and passaged to freshly coated plates every 7 days. Beginning in week two, a one-half media change was performed in between passages.

FIG. 5b is a set of representative flow cytometry plots showing the frequency of CD7+CD5+ proT cells and CD7+CD56+ NK cells after 7 days of culture in the presence or absence of IL-3, TNFα, or IL-3+TNFα. Frequencies are shown as mean±standard error from n=4 independent UCB donors.

FIG. 5c is a set of representative flow cytometry plots showing the frequency of CD7+CD5+ proT cells and CD7+CD56+ NK cells after 14 days of culture in the presence or absence of IL-3, TNFα, or IL-3+TNFα. Frequencies are shown as mean±standard error from n=4 independent UCB donors.

FIG. 5d is a set of graphs showing the fold expansion of total cells, CD7+CD5+ proT cells, CD7+CD56+ NK cells, and CD14/33+ myeloid cells after 7 days of culture in the presence or absence of IL-3, TNFα, or IL-3+TNFα. *p<0.05 from n=4 independent UCB donors.

FIG. 5e is a set of graphs showing the fold expansion of total cells, CD7+CD5+ proT cells, CD7+CD56+ NK cells, and CD14/33+ myeloid cells after 14 days of culture in the presence or absence of IL-3, TNFα, or IL-3+TNFα. *p<0.05 from n=4 independent UCB donors.

FIG. 6a is a set of representative histograms of CD117 (c-kit; SCF receptor), CD123 (IL-3 receptor), and CD127 (IL-7 receptor) expression on the surface of CD34+ HSPCs with or without TNFα stimulation for 24 hours.

FIG. 6b is a graph showing the frequencies of CD117+ cells, CD123+ cells, and CD127+ cells in the presence or absence of TNFα stimulation for 24 hours. *p<0.05 from n=3 independent UCB donors.

FIG. 6c is a graph showing the median fluorescent intensity (MFI) of CD117+ cells, CD123+ cells, and CD127+ cells in the presence or absence of TNFα stimulation for 24 hours. *p<0.05 from n=3 independent UCB donors.

FIG. 7a is a set of representative flow cytometry plots of CD7+CD5+ proT cells generated from CD34+ HSPCs after 14 days of culture in the presence or absence of IL-3+TNFα, and further in the presence or absence of VCAM-1. Frequencies are shown as mean±standard error from n=2 independent UCB donors.

FIG. 7b is a set of representative flow cytometry plots of CD7+CD1a+ preT cells generated from CD34+ HSPCs after 14 days of culture in the presence or absence of IL-3+TNFα, and further in the presence or absence of VCAM-1. Frequencies are shown as mean±standard error from n=2 independent UCB donors.

FIG. 7c is a set of representative flow cytometry plots of CD7+CD5+ proT cells and CD7+CD1a+ preT cells generated in the presence of IL-3+TNFα, and further in the presence of VCAM-1. Frequencies are shown as mean±standard error from n=2 independent UCB donors.

FIG. 7d is a set of representative flow cytometry plots of CD7+CD5+ proT cells and CD7+CD1a+ preT cells generated in the presence of IL-3+TNFα, but in the absence of VCAM-1. Frequencies are shown as mean±standard error from n=2 independent UCB donors.

FIG. 8 is a set of graphs showing the number of total cells, CD7+ lymphoid cells, and CD14/33+ myeloid cells in the presence or absence of TNFα, and further in the presence or absence of TPO. *p<0.05 for n=3 independent UCB donors; ns=not significant.

FIG. 9 is a schematic illustrating the non-limiting exemplary embodiments disclosed herein.

Other features and advantages of the disclosure will become apparent from the following detailed description and from the exemplary embodiments.

### DETAILED DESCRIPTION OF THE DISCLOSURE

Generally, the present disclosure provides a serum-free culture medium; a kit comprising the serum-free culture medium disclosed herein; a method of generating proT cells and derivatives of proT cells; a method of generating NK cells and derivatives of NK cells; a proT cell or a derivative thereof generated by the method disclosed herein; an NK cell or a derivative thereof generated by the method disclosed herein; a population of progenitor T cells generated by the method disclosed herein; a population of NK cells generated by the method disclosed herein; and methods for enhancing the immune response in a subject and for increasing the number of T cells or NK cells in a subject.

### Definitions

The terminology used herein is for the purpose of describing particular embodiments only and is not intended to be limiting of example embodiments of the invention. Unless defined otherwise, all technical and scientific terms used herein generally have the same meaning as commonly understood by one of ordinary skill in the art to which this disclosure belongs.

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrases “one or more” or “at least one” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” or “at least one” refers, whether related or unrelated to those elements specifically identified. Thus, as a non-limiting example, “one or more of A and B” (or, equivalently, “one or more of A or B,” or, equivalently “one or more of A and/or B”) or “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

A “subject” is a vertebrate, preferably a mammal (e.g., a non-human mammal), more preferably a primate and still more preferably a human. Mammals include, but are not limited to, primates, humans, farm animals, sport animals, and pets.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “treatment”, “treat” or “treating” or “amelioration” as used herein is an approach for obtaining beneficial or desired clinical results. For purposes of this disclosure, beneficial or desired clinical results include, but are not limited to, one or more of the following: increased immune response, increased T cell response, decreased extent of damage from a disease, condition, or disorder, decreased duration of a disease, condition, or disorder, and/or reduction in the number, extent, or duration of symptoms related to a disease, condition, or disorder. The term includes the administration of the compounds, agents, drugs or pharmaceutical compositions of the present disclosure to prevent or delay the onset of one or more symptoms, complications, or biochemical indicia of a disease or condition; lessening or improving one or more symptoms; shortening or reduction in duration of a symptom; or arresting or inhibiting further development of a disease, condition, or disorder. Treatment may be prophylactic (to prevent or delay the onset of a disease, condition, or disorder, or to prevent the manifestation of clinical or subclinical symptoms thereof) or therapeutic suppression or alleviation of symptoms after the manifestation of the disease, condition, or disorder.

As used herein, the term “stem cell” refers to a cell that can differentiate into more specialized cells and has the capacity for self-renewal. Stem cells include pluripotent stem cells (PSCs), such as embryonic stem cells (ESCs) and induced pluripotent stem cells (iPSCs), and multipotent stem cells, such as umbilical cord blood stem cells, and adult stem cells, which are found in various tissues. Methods for obtaining, deriving or producing stem cells are known in the art.

As used herein, the term “progenitor cell” refers to a cell that can differentiate into one or more types of cells, but typically has a limited capacity for self-renewal. Progenitor cells are derivatives of stem cells and have more limited potency relative to their corresponding source stem cells. For example, hematopoietic stem cells (HSCs), found in adult bone marrow, peripheral blood (in smaller numbers) and in umbilical cord blood, have the capacity to give rise to all other blood cells. Hematopoietic progenitor cells are multipotent or lineage-committed cells derived from HSCs that have the capacity to give rise to a more limited or specific type of blood cell. Hematopoietic stem and progenitor cells (HSPCs) typically exist as a heterogeneous population in vivo and have use as a heterogeneous population as described herein.

As used herein, the terms “progenitor T cell” and “proT cell” refer to a cell that is derived from a pluripotent stem cell or a CD34+ hematopoietic stem and/or progenitor cell and expresses at least CD7+, and has the capacity to differentiate into one or more types of immature and mature T cells. Examples of progenitor T cells include, but are not limited to, CD7+ cells, CD7+CD5+ cells, CD7+CD5+CD34+ cells, CD7+CD5+CD45RA+ cell, and/or CD7+CD5+CD1a+ cell.

As used herein, the terms “derivative of a progenitor T cell”, “derivative of a proT cell”, “progenitor T cell derivative” and “proT cell derivative” refer to any type of immature or mature T cell derived from a progenitor T cell. A mature T cell includes cells that express a combination of CD4, CD8 and CD3 cell surface markers. Examples of derivatives of progenitor T cells include, but are not limited to, double-positive CD4+CD8+ T cells, single-positive CD4+CD3+ cells, and single-positive CD8+CD3+ cells.

As used herein, a “defined culture medium” refers to a chemically-defined formulation comprised solely of chemically-defined constituents. A defined medium may include constituents having known chemical compositions. Medium constituents may be synthetic and/or derived from known non-synthetic sources. For example, a defined medium may include one or more growth factors secreted from known tissues or cells. However, the defined medium will not include the conditioned medium from a culture of such cells. A defined medium may include specific, known serum components isolated from an animal, including human serum components, but the defined medium will not include serum (i.e., a defined culture medium will always be serum-free). Any serum components provided in the defined medium such as, for example, bovine serum albumin (BSA), are preferably substantially homogeneous.

As used herein, “serum-free medium” refers to a cell culture medium that lacks animal serum. Serum-free medium may include specific, known serum components isolated from an animal (including human animals), such as, for example, BSA.

As used herein, “Delta-like-4”, “DL4” and “Notch ligand DL4” refer to a protein that in humans is encoded by the DLL4 gene. DL4 is a member of the Notch signaling pathway and is also referred to in the art as “Delta like ligand 4” and “DLL4”. Herein, reference to DL4 is not limited to the entire DL4 protein, but includes at least the signaling peptide portion of DL4. For example, a commercially available product (Sino Biologicals) comprising the extracellular domain (Met 1-Pro 524) of human DL4 (full-length DL4 accession number NP_061947.1; SEQ ID NO: 1) fused to the Fc region of human IgG₁ at the C-terminus is a DL4 protein suitable for use in the media formulations and methods provided herein.

As used herein, an “adhesion ligand” refers to any ligand capable of interacting with a transmembrane protein receptor to promote adhesion of a cell to the extracellular matrix (e.g., basal lamina) and/or to other cells. As used herein, an “integrin ligand” refers to any ligand capable of interacting with one or more types of integrins to promote adhesion of a cell to the extracellular matrix (e.g., basal lamina). Examples of adhesion ligands and/or integrin ligands include, but are not limited to VCAM-1, fibronectin and RetroNectin™.

As used herein, “Vascular cell adhesion molecule 1” and “VCAM-1” refer to a protein that in humans is encoded by the VCAM1 gene. VCAM-1 is a cell surface sialoglycoprotein, a type I membrane protein that is a member of the Ig superfamily. VCAM-1 is also referred to in the art as “vascular cell adhesion protein 1 and cluster of differentiation 106” (CD106). Herein, reference to VCAM-1 is not limited to the entire VCAM-1 protein, but includes at least the signaling peptide portion of VCAM-1 (QIDSPL (SEQ ID NO: 2) or TQIDSPLN (SEQ ID NO: 3)). For example, a commercially available mouse VCAM-1-Fc chimeric protein (R&D) that comprises (Phe25-Glu698) region of mouse VCAM-1 (full-length murine VCAM-1 accession number CAA47989; SEQ ID NO: 4) fused with the Fc region of human IgG₁ is a VCAM-1 protein suitable for use herein. Use of at least a portion of human VCAM-1 (full-length human VCAM-1 accession number P19320, NP001069, EAW72950; SEQ ID NO: 5) may also be suitable for use in the media formulations and methods provided herein.

As used herein, “tumor necrosis factor alpha” and “TNFα” refer to a protein that in humans is encoded by the TNFα gene. TNFα is a cytokine and is also referred to in the art as “TNF”, “tumor necrosis factor”, “cachectin”, “cachexin”, and “tumor necrosis factor ligand superfamily member 2”. Herein, reference to TNFα is not limited to the full-length TNFα protein, but is intended to encompass any functionally equivalent fragment of TNFα. For example, any functionally equivalent fragment of the full-length TNFα (accession number P01375; SEQ ID NO: 6) may be suitable for use in the media formulations and methods provided herein.

As used herein, “interleukin 3” and “IL-3” refer to a protein that in humans is encoded by the IL3 gene. IL-3 is a cytokine and is also referred to in the art as “hematopoietic growth factor”, “mast cell growth factor”, “MCGF”, “multipotential colony-stimulating factor”, and “P-cell-stimulating factor”. Herein, reference to IL-3 is not limited to the full-length IL-3 protein, but is intended to encompass any functionally equivalent fragment of IL-3. For example, any functionally equivalent fragment of the full-length IL-3 (accession number P08700; SEQ ID NO: 7) may be suitable for use in the media formulations and methods provided herein.

As used herein, a “functionally equivalent fragment” and a “functionally equivalent fragment thereof” refer to a fragment, portion, segment, or part of a full-length protein, wherein the fragment, portion, segment, or part retains at least one functional characteristic of the full-length protein. For example, a functionally equivalent fragment of human DL4 may be any fragment, portion, segment, or part of DL4 that comprises at least the extracellular domain (Met 1-Pro 524) of the human DL4 protein. A functionally equivalent fragment of VCAM-1 may be any fragment, portion, segment, or part of VCAM-1 that comprises at least the signaling peptide portion of the VCAM-1 protein (in particular, QIDSPL (SEQ ID NO: 2) or TQIDSPLN (SEQ ID NO: 3)). A functionally equivalent fragment of a cytokine may be any fragment, portion, segment, or part of a cytokine that retains the cell signaling activity of the full-length cytokine; and so on. In an embodiment, the “functionally equivalent fragment” and a “functionally equivalent fragment thereof” is mammalian, for example, mouse, rat or human.

### General Techniques

Unless otherwise defined herein, scientific and technical terms used in connection with the present disclosure shall have the meanings that are commonly understood by those of ordinary skill in the art. Generally, nomenclatures used in connection with, and techniques of, cell and tissue culture, molecular biology, immunology, microbiology, genetics and protein and nucleic acid chemistry and hybridization described herein are those well-known and commonly used in the art.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999); and Immunobiology (C. A. Janeway and P. Travers, 1997).

### Defined Culture Media Formulations

Generally, the defined culture medium provided herein is a serum-free culture medium which comprises TNFα, IL-3 and a Notch ligand, or functionally equivalent fragments thereof. The serum-free culture medium may further comprise at least one of: stem cell factor (SCF), interleukin 7 (IL-7) or FMS-like tyrosine kinase 3 ligand (FLT3L) or functionally equivalent fragments thereof. The serum-free culture medium may further comprise at least two of: SCF, IL-7 or FLT3L or functionally equivalent fragments thereof. The serum-free culture medium may further comprise SCF, IL-7 and FLT3L or functionally equivalent fragments thereof. The TNFα, IL-3, Notch ligand, SCF, IL-7 and/or FLT3L or functionally equivalent fragments thereof may be exogenously added to the culture medium or expressed in the culture medium using techniques known in the art.

The Notch ligand or the functionally equivalent fragment thereof may be adsorbed or immobilized to a substrate. In certain embodiments, the defined culture medium further comprises VCAM-1 or a functionally equivalent fragment thereof, which may also be adsorbed or immobilized to a substrate. In certain embodiments, other adhesion ligands and/or integrin ligands may be used in place of, or in addition to, VCAM-1. Examples of other adhesion ligands and/or integrin ligands that may be suitable for inclusion in the defined culture media formulations provided herein include, but are not limited to, fibronectin and RetroNectin™. In certain embodiments, the defined culture medium further comprises TPO or a functionally equivalent fragment thereof. The VCAM-1 and/or TPO or functionally equivalent fragments thereof may be exogenously added to the culture medium or expressed in the culture medium using techniques known in the art.

In various embodiments of the defined culture medium provided herein, the concentration of TNFα is between about 1 ng/ml and about 50 ng/ml. Suitable concentrations of TNFα in the medium include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of TNFα in the medium is about 10 ng/ml. In an embodiment, the concentration of TNFα in the medium is about 5 ng/ml.

In various embodiments of the defined culture medium provided herein, the concentration of IL-3 is between about 1 ng/ml and about 50 ng/ml. Suitable concentrations of IL-3 in the medium include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of IL-3 in the medium is about 10 ng/ml. In an embodiment, the concentration of IL-3 in the medium is about 5 ng/ml.

In various embodiments of the defined culture medium provided herein, the concentration of each one of SCF, IL-7 and FLT3L (and optionally, TPO) is between about 5 ng/ml and about 200 ng/ml. Suitable concentrations of SCF, IL-7 and FLT3L (and optionally, TPO) in the medium include, but are not limited to, about 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, and about 200 ng/ml. In an embodiment, the concentration of SCF, IL-7 and FLT3L (and optionally, TPO) in the medium is about 100 ng/ml. In an embodiment, the concentration of SCF, IL-7 and FLT3L (and optionally, TPO) in the medium is about 20 ng/ml.

The Notch ligand may be any ligand capable of binding to a Notch transmembrane receptor and activating the Notch signaling pathway. Preferably, the Notch ligand is capable of binding to at least the Notch 1 receptor, which is critical for T cell development. Optionally, the Notch ligand may be capable of binding to one or more other Notch receptors, for example, the Notch 3 receptor. In various embodiments of the defined culture medium, the Notch ligand may be DL4, DL1, JAG2, or a combination thereof. In an embodiment, the Notch ligand is DL4. In certain embodiments, the Notch ligand is adsorbed or immobilized to a substrate at a coating concentration of between about 0.5 ng/mm² and about 50 ng/mm². Suitable coating concentrations of the Notch ligand include, but are not limited to, about 50 ng/mm², 24 ng/mm², 12 ng/mm², 6 ng/mm², 5 ng/mm², 4 ng/mm², 3 ng/mm², 2 ng/mm², 1 ng/mm², 0.9 ng/mm², 0.8 ng/mm², 0.7 ng/mm², 0.6 ng/mm² and about 0.5 ng/mm². In an embodiment, the Notch ligand is adsorbed or immobilized to a substrate at a coating concentration of about 12 ng/mm². In an embodiment, the Notch ligand is adsorbed or immobilized to a substrate at a coating concentration of about 3.2 ng/mm². In an embodiment, the Notch ligand is adsorbed or immobilized to a substrate at a coating concentration of about 2 ng/mm². In an embodiment, the Notch ligand is adsorbed or immobilized to a substrate at a coating concentration of about 0.8 ng/mm².

Suitable substrates for adsorption or immobilization of the Notch ligand (and optionally, VCAM-1) include any means of support that provide sufficient resistance or tension for activating the Notch signaling pathway. In certain embodiments, the substrate may be one or more beads contained within the defined medium. In various embodiments, the substrate may be any container, vessel or receptacle suitable for culturing cells and/or tissues. Examples of suitable substrates include cell/tissue culture plates (including multiwell plates), petri dishes, bioreactors, culture bags, and the like, or any projection or protrusion extending into the container, vessel or receptacle therefrom. The adsorption or immobilization of the Notch ligand (and optionally, VCAM-1) to a substrate may be done by a variety of conjugation or tethering techniques, as known to those of ordinary skill in the art. Examples of such techniques include, but are not limited to, fragment crystallizable (Fc) region of an immunoglobulin molecule (such as, e.g., human IgG₁); biotin-streptavidin/neutravidin/avidin conjugation methods; and click chemistry conjugation methods.

In various embodiments, the defined culture medium provided herein may further comprise additional factors or additives that are suitable for culturing stem cells, progenitor cells, HSPCs, proT cells, preT cells and/or mature T cells. Examples of such additional factors or additives include, but are not limited to human serum albumin, bovine serum albumin, L-glutamine and/or other amino acids, insulin, transferrin, low-density lipoprotein, ascorbic acid, 2-mercaptoethanol, and antibiotics (e.g., penicillin and streptomycin).

### Method of Generating Progenitor T Cells

Generally, the in vitro method of generating proT cells and derivatives of proT cells (including preT cells, immature and mature T cells) comprises culturing stem and/or progenitor cells in a serum-free medium in the presence of TNFα, IL-3 and a Notch ligand or functionally equivalent fragments thereof. The serum-free culture medium may further comprise at least one of: SCF, IL-7 or FLT3L or functionally equivalent fragments thereof. The serum-free culture medium may further comprise at least two of: SCF, IL-7 or FLT3L or functionally equivalent fragments thereof. The serum-free culture medium may further comprise SCF, IL-7 and FLT3L or functionally equivalent fragments thereof. The Notch ligand or the functionally equivalent fragment thereof may be adsorbed or immobilized to a substrate. In certain embodiments, the culturing may further be in the presence of VCAM-1 or a functionally equivalent fragment thereof, which may also be adsorbed or immobilized to a substrate. In certain embodiments, other adhesion ligands and/or integrin ligands may be used in place of, or in addition to, VCAM-1. Examples of other adhesion ligands and/or integrin ligands that may be suitable for use in the method of generating proT cells provided herein include, but are not limited to, fibronectin and RetroNectin™. In certain embodiments, the culturing may further be in the presence of TPO or a functionally equivalent fragment thereof.

In the method of generating proT cells and derivatives of proT cells provided herein, TNFα is preferably provided to the cells in culture at day 0 or day 1. IL-3 may be provided to the cells in culture at the same time as TNFα or after TNFα has been provided, but preferably not before TNFα has been provided. In certain embodiments, IL-3 is provided to the cells in culture 1, 2, 3, 4 or 5 days after TNFα has been provided to the cells.

In various embodiments of the method of generating proT cells and derivatives of proT cells provided herein, the concentration of TNFα in the culture is between about 1 ng/ml and about 50 ng/ml. Suitable concentrations of TNFα in the culture include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of TNFα in the culture is about 10 ng/ml. In an embodiment, the concentration of TNFα in the culture is about 5 ng/ml.

In various embodiments of the method of generating proT cells and derivatives of proT cells provided herein, the concentration of IL-3 in the culture is between about 1 ng/ml and about 50 ng/ml. Suitable concentrations of IL-3 in the culture include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of IL-3 in the culture is about 10 ng/ml. In an embodiment, the concentration of IL-3 in the culture is about 5 ng/ml.

In various embodiments of the method of generating proT cells and derivatives of proT cells provided herein, the concentration of each one of SCF, IL-7 and FLT3L (and optionally, TPO) in the culture is between about 5 ng/ml and about 200 ng/ml. Suitable concentrations of SCF, IL-7 and FLT3L (and optionally, TPO) in the culture include, but are not limited to, about 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, and about 200 ng/ml. In an embodiment, the concentration of SCF, IL-7 and FLT3L (and optionally, TPO) in the culture is about 100 ng/ml. In an embodiment, the concentration of SCF, IL-7 and FLT3L (and optionally, TPO) in the culture is about 20 ng/ml.

The Notch ligand may be any ligand capable of binding to a Notch transmembrane receptor and activating the Notch signaling pathway. Preferably, the Notch ligand is capable of binding to at least the Notch 1 receptor, which is critical for T cell development. Optionally, the Notch ligand may be capable of binding to one or more other Notch receptors, for example, the Notch 3 receptor. In various embodiments of the method of generating proT cells and derivatives of proT cells provided herein, the Notch ligand may be DL4, DL1, JAG2, or a combination thereof. In an embodiment, the Notch ligand is DL4. In certain embodiments, the Notch ligand is adsorbed or immobilized to a substrate at a coating concentration of between about 0.5 ng/mm² and about 50 ng/mm². Suitable coating concentrations of the Notch ligand include, but are not limited to, about 50 ng/mm², 24 ng/mm², 12 ng/mm², 6 ng/mm², 5 ng/mm², 4 ng/mm², 3 ng/mm², 2 ng/mm², 1 ng/mm², 0.9 ng/mm², 0.8 ng/mm², 0.7 ng/mm², 0.6 ng/mm² and about 0.5 ng/mm². In an embodiment, the Notch ligand is adsorbed or immobilized to a substrate at a coating concentration of about 12 ng/mm². In an embodiment, the Notch ligand is adsorbed or immobilized to a substrate at a coating concentration of about 3.2 ng/mm². In an embodiment, the Notch ligand is adsorbed or immobilized to a substrate at a coating concentration of about 2 ng/mm². In an embodiment, the Notch ligand is adsorbed or immobilized to a substrate at a coating concentration of about 0.8 ng/mm².

Suitable substrates for adsorption or immobilization of the Notch ligand (and optionally, VCAM-1 and/or another adhesion ligand and/or integrin ligand) include any means of support that provide sufficient resistance or tension for activating the Notch signaling pathway. In certain embodiments, the substrate may be one or more beads contained within the defined culture medium. In various embodiments, the substrate may be any container, vessel or receptacle suitable for culturing cells and/or tissues. Examples of suitable substrates include cell/tissue culture plates (including multiwell plates), petri dishes, culture bags, and the like, or any projection or protrusion extending into the container, vessel or receptacle therefrom. The adsorption or immobilization of the Notch ligand (and optionally, VCAM-1 and/or another adhesion ligand and/or integrin ligand) to a substrate may be done by a variety of conjugation or tethering techniques, as known to those of ordinary skill in the art. Examples of such techniques include, but are not limited to, fragment crystallizable (Fc) region of an immunoglobulin molecule (such as, e.g., human IgG₁); biotin-streptavidin/neutravidin/avidin conjugation methods; and click chemistry conjugation methods.

In certain embodiments, the stem and/or progenitor cells are pluripotent stem cells, such as ESCs or iPSCs. In an embodiment, the stem and/or progenitor cells are CD34+ HSPCs. The HSPCs may be derived from primary tissues, such as cord blood, peripheral blood or bone marrow. The HSPCs may also be derived in vitro from ESCs, iPSCs or other intermediate stem cells. In certain embodiments, the stem and/or progenitor cells are human cells.

In an embodiment, the method is performed in a two-dimensional (2D) culture system. For example, one or more wells of a standard tissue culture plate are coated with a Notch ligand and optionally, VCAM-1. In an embodiment, the Notch ligand (and optionally VCAM-1) are provided as adsorbed proteins. Stem cells and/or progenitor cells are then seeded into the 2D Notch ligand- (and optionally VCAM-1-) coated wells in serum-free medium and cultured for a time and under conditions suitable for generating proT cells and/or derivatives of proT cells.

In an embodiment, wells of a standard 96-well tissue culture plate are coated overnight with about 50 μL/well of DL4-Fc (and optionally, VCAM-1-Fc). Coated wells are then washed to remove unbound ligand and seeded with stem and/or progenitor cells in a defined medium at a suitable plating density. In an embodiment, the defined medium is, for example, Iscove's Modified Dulbecco's Medium with 20% bovine serum albumin, insulin, and transferrin serum substitute (IMDM+BIT) supplemented with TNFα, IL-3 and a Notch ligand or functionally equivalent fragments thereof, and optionally SCF, IL-7 and/or FLT3L or functionally equivalent fragments thereof. The seeded cells are cultured at an appropriate temperature, e.g., 37° C., and for a time sufficient to generate proT cells and derivatives of proT cells, such as, for example, at least 7 days or at least 14 days.

In certain embodiments, the methods of generating proT cells and proT cell derivatives provided herein may further include genetic modification or gene editing of the cells. The genetic modification or gene editing may be carried out before, after, or at any step during the methods provided herein. Methods of genetic modification, engineering or editing are known to those of ordinary skill in the art. Examples include, but are not limited to: random mutagenesis; targeted mutagenesis using recombinant DNA techniques, site-specific nucleases and the like; zinc-finger nucleases (ZFNs); transcription activator-like effector nucleases (TALENs); and clustered regularly interspaced short palindromic repeats (CRISPR/Cas9) technology. In certain embodiments, the genetically modified, engineered or edited T cells may include chimeric antigen receptor T cells (CAR-T) or modified T cell receptor T cells (TCR-T).

To confirm generation of proT cells and derivatives of proT cells (including preT cells, immature and mature T cells), the cells may be analyzed for one or more features indicative of proT, preT or mature T cells, such as, for example, one or more cell surface markers. Suitable techniques for analyzing cell surface markers are known to those of ordinary skill in the art, and may include, for example, flow cytometry as used herein, or immunocytochemistry.

### Method of Generating NK Cells

Generally, the in vitro method of generating NK and derivatives of NK cells comprises culturing stem and/or progenitor cells in a serum-free medium in the presence of TNFα, IL-3 and a Notch ligand or functionally equivalent fragments thereof. The serum-free culture medium may further comprise at least one of: SCF, IL-7 or FLT3L, or functionally equivalent fragments thereof. The serum-free culture medium may further comprise at least two of: SCF, IL-7 or FLT3L, or functionally equivalent fragments thereof. The serum-free culture medium may further comprise SCF, IL-7 and FLT3L or functionally equivalent fragments thereof. The Notch ligand or the functionally equivalent fragment thereof may be adsorbed or immobilized to a substrate. In certain embodiments, the culturing may further be in the presence of VCAM-1 or a functionally equivalent fragment thereof, which may also be adsorbed or immobilized to a substrate. In certain embodiments, other adhesion ligands and/or integrin ligands may be used in place of, or in addition to, VCAM-1. Examples of other adhesion ligands and/or integrin ligands that may be suitable for use in the method of generating NK cells provided herein include, but are not limited to, fibronectin and RetroNectin™. In certain embodiments, the culturing may further be in the presence of TPO or a functionally equivalent fragment thereof.

In the method of generating NK cells and derivatives of NK cells provided herein, TNFα is preferably provided to the cells in culture at day 0 or day 1. IL-3 may be provided to the cells in culture at the same time as TNFα or after TNFα has been provided, but preferably not before TNFα has been provided. In certain embodiments, IL-3 is provided to the cells in culture 1, 2, 3, 4 or 5 days after TNFα has been provided to the cells.

In various embodiments of the method of generating NK cells and derivatives of NK cells provided herein, the concentration of TNFα in the culture is between about 1 ng/ml and about 50 ng/ml. Suitable concentrations of TNFα in the culture include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of TNFα in the culture is about 10 ng/ml. In an embodiment, the concentration of TNFα in the culture is about 5 ng/ml.

In various embodiments of the method of generating NK cells and derivatives of NK cells provided herein, the concentration of IL-3 in the culture is between about 1 ng/ml and about 50 ng/ml. Suitable concentrations of IL-3 in the culture include, but are not limited to, about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml and about 50 ng/ml. In an embodiment, the concentration of IL-3 in the culture is about 10 ng/ml. In an embodiment, the concentration of IL-3 in the culture is about 5 ng/ml.

In various embodiments of the method of generating NK cells and derivatives of NK cells provided herein, the concentration of each one of SCF, IL-7 and FLT3L (and optionally, TPO) in the culture is between about 5 ng/ml and about 200 ng/ml. Suitable concentrations of SCF, IL-7 and FLT3L (and optionally, TPO) in the culture include, but are not limited to, about 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, and about 200 ng/ml. In an embodiment, the concentration of SCF, IL-7 and FLT3L (and optionally, TPO) in the culture is about 100 ng/ml. In an embodiment, the concentration of SCF, IL-7 and FLT3L (and optionally, TPO) in the culture is about 20 ng/ml.

The Notch ligand may be any ligand capable of binding to a Notch transmembrane receptor and activating the Notch signaling pathway. Preferably, the Notch ligand is capable of binding to at least the Notch 1 receptor. Optionally, the Notch ligand may be capable of binding to one or more other Notch receptors, for example, the Notch 3 receptor. In various embodiments of the method of generating NK cells and derivatives of NK cells provided herein, the Notch ligand may be DL4, DL1, JAG2, or a combination thereof. In an embodiment, the Notch ligand is DL4. In certain embodiments, the Notch ligand is adsorbed or immobilized to a substrate at a coating concentration of between about 0.5 ng/mm² and about 50 ng/mm². Suitable coating concentrations of the Notch ligand include, but are not limited to, about 50 ng/mm², 24 ng/mm², 12 ng/mm², 6 ng/mm², 5 ng/mm², 4 ng/mm², 3 ng/mm², 2 ng/mm², 1 ng/mm², 0.9 ng/mm², 0.8 ng/mm², 0.7 ng/mm², 0.6 ng/mm² and about 0.5 ng/mm². In an embodiment, the Notch ligand is adsorbed or immobilized to a substrate at a coating concentration of about 12 ng/mm². In an embodiment, the Notch ligand is adsorbed or immobilized to a substrate at a coating concentration of about 3.2 ng/mm². In an embodiment, the Notch ligand is adsorbed or immobilized to a substrate at a coating concentration of about 2 ng/mm². In an embodiment, the Notch ligand is adsorbed or immobilized to a substrate at a coating concentration of about 0.8 ng/mm².

Suitable substrates for adsorption or immobilization of the Notch ligand (and optionally, VCAM-1 and/or another adhesion ligand and/or integrin ligand) include any means of support that provide sufficient resistance or tension for activating the Notch signaling pathway.

In certain embodiments, the stem and/or progenitor cells are pluripotent stem cells, such as ESCs or iPSCs. In an embodiment, the stem and/or progenitor cells are CD34+ HSPCs. The HSPCs may be derived from primary tissues, such as cord blood, peripheral blood or bone marrow. The HSPCs may also be derived in vitro from ESCs, iPSCs or other intermediate stem cells. In certain embodiments, the stem and/or progenitor cells are human cells.

In an embodiment, the method is performed in a two-dimensional (2D) culture system. For example, one or more wells of a standard tissue culture plate are coated with a Notch ligand and optionally, VCAM-1. In an embodiment, the Notch ligand (and optionally VCAM-1) are provided as adsorbed proteins. Stem cells and/or progenitor cells are then seeded into the 2D Notch ligand- (and optionally VCAM-1-) coated wells in serum-free medium and cultured for a time and under conditions suitable for generating NK cells and/or derivatives of NK cells.

In an embodiment, wells of a standard 96-well tissue culture plate are coated overnight with about 50 μL/well of DL4-Fc (and optionally, VCAM-1-Fc). Coated wells are then washed to remove unbound ligand and seeded with stem and/or progenitor cells in a defined medium at a suitable plating density. In an embodiment, the defined medium is, for example, Iscove's Modified Dulbecco's Medium with 20% bovine serum albumin, insulin, and transferrin serum substitute (IMDM+BIT) supplemented with TNFα, IL-3 and a Notch ligand or functionally equivalent fragments thereof and optionally SCF, IL-7 and/or FLT3L or functionally equivalent fragments thereof. The seeded cells are cultured at an appropriate temperature, e.g., 37° C., and for a time sufficient to generate NK cells and derivatives of NK cells, such as, for example, at least 7 days or at least 14 days.

In certain embodiments, the methods of generating NK cells and NK cell derivatives provided herein may further include genetic modification or gene editing of the cells, for example, as described herein. The genetic modification or gene editing may be carried out before, after, or at any step during the methods provided herein.

To confirm generation of NK cells and derivatives of NK cells, the cells may be analyzed for one or more features indicative of NK cells, such as, for example, one or more cell surface markers. Suitable techniques for analyzing cell surface markers are known to those of ordinary skill in the art, and may include, for example, flow cytometry as used herein, or immunocytochemistry.

### Progenitor T Cells and Derivatives thereof Generated using the Defined Media and the Methods Provided Herein

The present disclosure provides proT cells and derivatives of proT cells, including preT cells, immature and mature T cells, and/or a population of pro T cells generated using the defined media and/or methods provided herein. Preferably, the proT cells are human. In an embodiment, the human proT cells may be characterized phenotypically via expression of CD4 and CD8 on the cell surface progressing via successive double-negative (DN; CD4−CD8−) stages: CD7+CD34+ primitive progenitor T cells followed by CD7+ and/or CD34− and/or CD5+ and/or CD45RA+ and/or CD1a+ proT cells and finally maturing to double-positive (DP; CD4+CD8+) and single-positive (SP; CD4+CD3+ or CD8+CD3+) T cells. In an embodiment, the human proT cells provided herein may be characterized by CD7 expression. In general, lymphoid cells may be identified by their small and round morphology and by blue colour in a Giemsa stain. In an embodiment, the proT cells provided herein may be functionally characterized. For example, CD7+ proT cell transplantation in vivo should result in the transplanted cells homing to the thymus, engrafting in the thymus, and then rapidly dividing to generate DP and SP T cells.

In various embodiments, the proT cells generated using the defined media and/or methods provided herein may be, for example, CD7+ cells, CD7+CD5+ cells, CD7+CD5+CD34+cells, CD7+CD5+CD45RA+ cell, and/or CD7+CD5+CD1a+ preT cells. In various embodiments, the proT cells are, for example proT1 (CD34+CD7+CD5−) and proT2 (CD34+CD7+CD5+) cells.

In various embodiments, the derivatives of proT cells generated using the defined media and/or methods provided herein may be, for example, double-positive CD4+CD8+ and/or single-positive CD4+CD3+ mature T cells and/or single-positive CD8+CD3+ mature T cells.

In certain embodiments, the proT cells and derivatives of proT cells generated using the defined media and/or the methods provided herein have a higher cell surface density of IL-3 receptors (CD123) compared to proT cells generated by culturing in the absence of TNFα.

In certain embodiments, the pluripotent stem cells, the CD34+ HSPCs, the CD7+ proT cells, the CD7+CD5+ proT cells, the CD7+CD5+CD34+ proT cells, the CD7+CD5+CD45RA+ proT cells, the CD7+CD5+CD1a+ preT cells, the CD4+CD8+ T cells, the CD4+CD3+ T cells and/or the CD8+CD3+ T cells may be genetically modified, engineered, or edited. The genetic modification, engineering or editing may be carried out before, after, or at any step during the method of generating the proT cells and the proT cell derivatives provided herein. Methods of genetic modification, engineering or editing are known to those of ordinary skill in the art. Examples include, but are not limited to: random mutagenesis; targeted mutagenesis using recombinant DNA techniques, site-specific nucleases and the like; zinc-finger nucleases (ZFNs); transcription activator-like effector nucleases (TALENs); and clustered regularly interspaced short palindromic repeats (CRISPR/Cas9) technology. In certain embodiments, the genetically modified, engineered or edited T cells may include chimeric antigen receptor T cells (CAR-T) or modified T cell receptor T cells (TCR-T).

In an embodiment, the proT cells and derivatives of proT cells generated using the defined media and/or the methods provided herein are autologous.

In an embodiment, the proT cells and derivatives of proT cells generated using the defined media and/or the methods provided herein are allogeneic.

### NK Cells and Derivatives thereof Generated using the Defined Media and the Methods Provided Herein

The present disclosure provides NK and derivatives of NK cells, and/or a population of NK cells generated using the defined media and/or methods provided herein. Preferably, the NK cells are human. In an embodiment, the human NK cells may be characterized phenotypically via expression of markers. In an embodiment, the NK cells provided herein may be functionally characterized using techniques known in the art.

In various embodiments, the NK cells generated using the defined media and/or methods provided herein may be, for example, CD7+CD56+ cells.

In certain embodiments, the NK cells may be genetically modified, engineered, or edited, for example, as described herein. The genetic modification, engineering or editing may be carried out before, after, or at any step during the method of generating the NK cells and the NK cell derivatives provided herein.

In an embodiment, the NK cells and derivatives of NK cells generated using the defined media and/or the methods provided herein are autologous.

In an embodiment, the NK cells and derivatives of NK cells generated using the defined media and/or the methods provided herein are allogeneic.

### Methods of Treatment With Progenitor T Cells and/or Derivatives Thereof and/or With NK Cells and/or Derivatives Thereof

In various embodiments, the proT cells and/or derivatives of proT cells disclosed herein may be used for enhancing the immune response in a subject and/or for increasing the number of T cells or in a subject or patient. In an embodiment a population of pro T cells and/or derivatives thereof are produced and administered to the subject or patient. In various embodiments, the NK cells and/or derivatives of NK cells disclosed herein may be used for enhancing the immune response in a subject and/or for increasing the number of NK cells or in a subject or patient. In an embodiment a population of NK cells and/or derivatives thereof are produced and administered to the subject or patient. Suitable subjects include subjects suffering from an immune deficiency, such as a T cell deficiency or an NK deficiency. The immune deficiency bay be caused by a medical condition, a chemical exposure, a radiation exposure or a combination thereof. Medical conditions which may be treated with the proT cells and/or derivatives of proT cells disclosed herein or with the NK cells and/or derivatives of NK cells disclosed herein comprise, but are not limited to, a cancer, a bone marrow failure, an anemia, a primary immunodeficiency disorder, an autoimmune disease, a partial thymectomy, an organ transplant, a viral infection (for example, HIV infection), a bacterial infection, a fungal infection and/or idiopathic CD4+ T-lymphocytopenia. Subjects or patients suffering from the effects of a chemical exposure who may be candidates for treatment with the proT cells and/or derivatives of proT cells or with the NK cells and/or derivatives of the NK cells disclosed herein include, for example, chemotherapy patients, anti-inflammatory drug therapy patients, subjects exposed to workplace-related chemicals or subjects suffering from an accidental poisoning with a chemical substance. Subjects suffering from the effects of a radiation exposure who may be candidates for treatment with the proT cells and/or derivatives of proT cells or with the NK cells and/or derivatives of the NK cells disclosed herein include, for example, radiotherapy patients, subjects exposed to workplace-related radioisotopes, subjects suffering from accidental poisoning with a radioisotope, or subjects exposed to nuclear meltdown or nuclear fallout.

It is contemplated that the allogeneic proT cells provided herein could be transferred to an irradiated subject in need of proT cells irrespective of major histocompatibility complex (MHC) disparities. Without being bound by theory, it is thought that proT cells, unlike mature T cells, do not cause graft versus host disease (GVHD), at least because proT cell precursors complete their differentiation in the thymus, where they become restricted to host MHC and yield T lymphocytes that are host tolerant. Thus, strict histocompatibility would not be required in therapeutic use of the proT cells provided herein.

The cells provided herein may be used, for example, to treat a subject in need of proT cells and/or more mature T cells or in need of NK cells. “Treat”, as used herein, refers to administering to the subject an effective amount of cells, as provided herein, under conditions suitable for increasing the number of T cells or NK cells in the subject, which may result in prevention, inhibition and/or therapeutic treatment of a medical condition associated with insufficient T cells or with insufficient NK cells. “Effective amount”, as used herein, refers to a therapeutically effective amount such as, for example, the amount of cells that, upon administration to a subject, is sufficient to achieve the intended purpose (e.g., treatment). The effective amount may vary from one subject to another and may depend upon one or more factors, such as, for example, subject gender, age, body weight, subject's health history, and/or the underlying cause of the condition to be prevented, inhibited and/or treated.

For example, subjects afflicted with a medical condition causing or resulting in lymphopenia may benefit from administration of a proT transplant as described herein. For example, subjects who are post-chemotherapy and/or post-irradiation, such as those receiving treatment for cancer, subjects having HIV infection, partial thymectomy, autoimmune diseases, such as lupus or rheumatoid arthritis, or diabetes may benefit from administration of the proT cells provided herein. In an embodiment, the cells provided herein may be used to induce host tolerance upon organ transplant.

### Kits for Generating Progenitor T Cells or NK Cells

The present disclosure provides kits for carrying out the methods of generating proT cells and/or derivatives of proT cells provided herein or for generating NK cells and/or derivatives of NK cells provided herein. Such kits typically comprise two or more components required for generation of proT cells or NK cells. Components of the kit include, but are not limited to, one or more of compounds, reagents, containers, equipment and instructions for using the kit. Accordingly, the methods described herein may be performed by utilizing pre-packaged kits provided herein.

In an embodiment, a kit comprising the defined serum-free culture medium disclosed herein is provided. The kit further comprises one or more of compounds, reagents, containers, equipment and instructions for using the kit.

In an embodiment, a kit comprising two types of media is provided. The two types of media are:
a. a serum-free culture medium comprising TNFα, IL-3, or functionally equivalent fragments thereof; and
b. a coating medium comprising a Notch ligand or a functionally equivalent fragment thereof.

In certain embodiments of the kit, the serum-free culture medium further comprises at least one of: SCF, IL-7 or FLT3L, or functionally equivalent fragments thereof. In certain embodiments of the kit, the serum-free culture medium further comprises at least two of: SCF, IL-7 or FLT3L, or functionally equivalent fragments thereof. In certain embodiments of the kit, the serum-free culture medium further comprises SCF, IL-7 and FLT3L, or functionally equivalent fragments thereof.

In certain embodiments of the kit, the serum-free culture medium further comprises TPO or a functionally equivalent fragment thereof.

In certain embodiments, the Notch ligand in the coating medium of the kit is DL4, DL1, JAG2, or a combination thereof. In certain embodiments of the kit, the coating medium further comprises VCAM-1 or a functionally equivalent fragment thereof. In certain embodiments, other adhesion ligands and/or integrin ligands may be used in place of, or in addition to, VCAM-1. Examples of other adhesion ligands and/or integrin ligands that may be suitable for inclusion in the kit provided herein include, but are not limited to, fibronectin and RetroNectin™.

The coating medium may be used to coat a suitable substrate (as described herein) with the Notch ligand and optionally, VCAM-1 and/or another adhesion ligand and/or integrin ligand. Subsequently, the serum-free culture medium may be used for culturing stem and/or progenitor cells in the presence of the Notch ligand- (and optionally VCAM-1-) coated substrate to generate proT cells and derivatives of proT cells or to generate NK cells and derivatives of NK cells.

In some embodiments, instructions for use of the kit to generate proT cells and/or derivatives of proT cells or to generate NK cells and/or derivatives of NK cells from stem and/or progenitor cells, such as PSCs or HSPCs, in vitro are provided. The instructions may comprise one or more protocols for: storing, preparing and using the coating medium and the serum-free medium contained in the kit; culture conditions, such as time, temperature, and/or gas incubation concentrations; harvesting protocols; and protocols for identifying proT cells and, optionally, derivatives of proT cells, such as preT cells and mature T cells or for identifying NK cells and, optionally, derivatives of NK cells. The kit may further include materials useful for conducting the present method such as, for example, cell/tissue culture plates (including multiwell plates), petri dishes, culture bags, and the like.

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Example 1: Materials and Methods

### DL4 Production and DL4+VCAM-1 Plate Coating

Recombinant DL4-Fc fusion protein was purchased from Sino Biological or manufactured in-house using HEK-293T cells and purified with HiTrap Protein G affinity columns (GE Healthcare) as previously described.⁶

Tissue culture 96-well plates were coated with DL4-Fc and VCAM-1-Fc (R&D Systems) overnight at 4° C. or for 3-4 hours at room temperature. To coat, DL4 and VCAM-1 were diluted to 15 μg/ml and 2.5 μg/ml, respectively, in 50 μl of phosphate-buffered saline (PBS), resulting in a coating concentration of approximately 24 ng/mm2 of DL4-Fc and 4 ng/mm2 of VCAM-1-Fc. For titration experiments, the concentration of DL4 or VCAM-1 was adjusted accordingly while maintaining a 50 μl volume of PBS. Experiments using 384-well plates scaled were coated similarly but with 16 μl of PBS. Wells were washed once with PBS prior to seeding cells to remove unbound DL4-Fc protein that inhibits Notch signaling.⁶

### Human CD34+ HSPC Enrichment from Umbilical Cord Blood

Umbilical cord blood (UCB) was collected from consenting donors at Mount Sinai Hospital, Toronto, Ontario, in accordance with institutional research ethics board policies. Mononuclear cells were fractioned via centrifugation using Lympholyte cell separation media (Cedarlane) and red blood cells were lysed using ammonium-chloride-potassium (ACK) lysing buffer prepared in-house. Mononuclear cells were enriched for >90% CD34+ cells using EasySep™ Human CD34+ Positive Selection Kit (Stemcell Technologies) according to the manufacturer's instructions. For certain experiments, enriched CD34+ cells were stained with CD34-APC and CD38-PE antibodies and sorted into CD34+CD38lo/− and CD34+CD38+ fractions using FACS Aria flow cytometer (Beckman Coulter).

### HSPC Culture on DL4+VCAM-1

For experiments 7 days or longer, HSPCs were seeded at 63-125 cells/mm² (equivalent to 2000-4000 cells/well of a 96-well plate) on DL4+VCAM-1-coated surfaces. Experiments shorter than 7 days (CFSE and qPCR) were seeded at 470-780 cells/mm² (equivalent to 15,000-25,000 cells/well of a 96-well plate) to provide enough cells for analysis. Cells were cultured in 100 μl/well (32 μl/well for 384-well plate) of Iscove's Modified Eagle Medium (IMDM) supplemented with 20% bovine serum albumin, insulin, and transferrin (BIT 9500) serum substitute (Stemcell Technologies), 1 μg/ml low-density lipoprotein (LDL; Stemcell Technologies), 60 μM ascorbic acid (Sigma), 24 μM 2-mercaptoethanol (Sigma), and 1% penicillin-streptomycin (P/S; Invitrogen). The cytokines used in control condition were SCF, FLT3L, TPO, and IL-7 (also known as the “4F cytokines”, all of which were obtained from R&D Systems), each at a concentration of 20 ng/ml or 100 ng/ml as indicated in the text. All other cytokines were purchased from R&D Systems and used in the combinations and concentrations indicated in the text. For certain experiments, the γ-secretase inhibitor (2S)-N-[(3,5-Difluorophenyl)acetyl]-L-alanyl-2-phenyl] glycine 1,1-dimethylethyl ester (DAPT) or dimethyl sulfoxide (DMSO) was added to media at the concentrations indicated in the text.

### Flow Cytometry

Prior to analysis via flow cytometry, adherent cells were collected from DL4+VCAM-1 surfaces using vigorous pipetting or TrypLE Express Enzyme (ThermoFisher) for 5 minutes at 37° C. Afterwards, cells were rinsed twice with Hanks Balanced Salt Solution (ThermoFisher) plus 2% FBS (HF). Surface antibodies were stained at 1:100-1:300 in HF at room temperature for 15 minutes. Cells were then rinsed once with HF and resuspended in HF with 1:1000 7-AAD to exclude dead cells. Data was acquired with a BD Fortessa™ and compensation and gating was performed with FlowJo™ software and further analysis with the R programming language (version 3.3.2). Antibodies used for the flow cytometry experiments are shown in Table 1.

TABLE 1
Antibodies

| | | | | Catalog |
|---|---|---|---|---|
| Antibody | Reactivity | Fluorophore | Source | Number |
| 7-AAD | Mouse/ | PerCP-Cy5-5 | Life Technologies | A1310 |
| | Human | | | |
| CD7 | Human | APC | BD Biosciences | 561604 |
| CD5 | Human | PECy7 | eBioscience | 25-0059-42 |
| CD34 | Human | PE | BD Biosciences | 555822 |
| CD34 | Human | APC | BD Biosciences | 555824 |
| CD38 | Human | BV605 | BD Biosciences | 562665 |
| CD38 | Human | PE | BD Biosciences | 555460 |
| CD14 | Human | FITC | BD Biosciences | 555397 |
| CD33 | Human | FITC | Biolegend | 303304 |
| CD1a | Human | BV421 | BD Biosciences | 563938 |
| CD56 | Human | BV605 | BD Biosciences | 562780 |
| CD117 | Human | Alexa- | Biolegend | 313233 |
| | | Fluor-488 | | |
| CD123 | Human | BV421 | BD Biosciences | 562517 |
| CD127 | Human | PE-Cy7 | Biolegend | 351320 |

### Proliferation Assays

To track the number of cell divisions, cells were stained with 2.5 μM CellTrace™ CFSE (ThermoFisher) in PBS and incubated at 37° C. for 8 minutes. CFSE was quenched by adding 5 times the volume of IMDM+BIT and incubating at 37° C. for 5 minutes. Cells were then resuspended in fresh media and seeded on DL4+VCAM-1 for culture. For analysis, cells were collected and stained for CD34, CD7, and CD5 surface markers as described above and analyzed using flow cytometry. Proliferation was modelled using FlowJo™ software and further analysis was performed with R.

### Quantitative Real-Time PCR

Cells were lysed and RNA isolated using the PureLink™ RNA Micro Kit (Invitrogen) according to the manufacturer's protocol. cDNA was reverse-transcribed from RNA using SuperScript™ III Reverse Transcriptase (Invitrogen) according to the manufacturer's protocol. cDNA was amplified with primers and FastStart SYBR Green Mastermix (Roche) using QuantStudio™ 6 Flex (Applied Biosystems). Relative expression of individual genes was calculated by the delta cycle threshold (ΔCt) method and normalization using β-actin to control for differences in input cDNA. PCR primer sequences are shown in Table 2.

TABLE 2
qRT-PCR Primer Sequence

| Target | Species | Forward Primer | Reverse Primer |
|---|---|---|---|
| ACTB | Human | CATGTACGTTGCTATCCAGGC | CTCCTTAATGTCACGCACGAT |
| | | (SEQ ID NO: 8) | (SEQ ID NO: 9) |
| BCL11B | Human | TCCAGCTACATTTGCACAACA | GCTCCAGGTAGATGCGGAAG |
| | | (SEQ ID NO: 10) | (SEQ ID NO: 11) |
| DTX1 | Human | ATCGGAGAAGGCTCTACAGG | CGTCTGGCCTCCTTTCTAACT |
| | | (SEQ ID NO: 12) | (SEQ ID NO: 13) |
| E2A | Human | CCGACTCCTACAGTGGGCTA | CGCTGACGTGTTCTCCTCG |
| | | (SEQ ID NO: 14) | (SEQ ID NO: 15) |
| GATA3 | Human | GTTGGCCTAAGGTGGTTGTG | ACAGGCTGCAGGAATAGGGA |
| | | (SEQ ID NO: 16) | (SEQ ID NO: 17) |
| HES1 | Human | CCTGTCATCCCCGTCTACAC | CACATGGAGTCCGCCGTAA |
| | | (SEQ ID NO: 18) | (SEQ ID NO: 19) |
| NOTCH1 | Human | GAGGCGTGGCAGACTATGC | CTTGTACTCCGTCAGCGTGA |
| | | (SEQ ID NO: 20) | (SEQ ID NO: 21) |
| SPI1 | Human | TGCAATGTCAAGGGAGGGGG | AAACCCTTCCATTTTGCACGC |
| | | (SEQ ID NO: 22) | (SEQ ID NO: 23) |
| TCF7 | Human | TGCACATGCAGCTATACCCAG | TGGTGGATTCTTGGTGCTTTTC |
| | | (SEQ ID NO: 24) | (SEQ ID NO: 25) |
| CEBPA | Human | GGAGCTGAGATCCCGACA | TTCTAAGGACAGGCGTGGAG |
| | | (SEQ ID NO: 26) | (SEQ ID NO: 27) |

### Cytokine Screens

For screening experiments, a Definitive Screening Design (DSD)⁷ was employed where each factor was tested at three levels (scaled to [−1,0,1]). The design included at least 2m+1 runs, where m is the number of factors, comprised of m fold-over pairs plus a center run (with all factors at level 0) and with orthogonal blocking of UCB donors.⁸

Absolute cell numbers were acquired for each cell population of interest and, for initial experiments, a z-score was calculated relative to the control condition:

$$z score = \frac{test - c o n t r o l}{S E ( control )}$$

where SE(⋅) is the standard error. Control conditions were the cytokines used previously in the DL4+VCAM-1 assay as in Shukla et al.⁶ Stepwise regression using the minimum Bayesian information criterion method was used to determine significant terms and, from this, a linear regression model of z-scores was constructed that included main and quadratic effects and two-factor interactions. This provided a better- or worse-than estimate for each factor compared to the control. The DSD layout and regression model were constructed using JMP 13 software (SAS). Follow up experiments measuring the dose response of top factors, the regression model was fit to the absolute cell count. Values were square root or log-transformed as necessary to ensure that residuals were approximately normal before fitting the regression model.

Test conditions were arranged using serial dilutions of each cytokine and laid out using an Eppendorf epMotion 5070 liquid handler in 384-well plates previously coated with DL4 and VCAM-1.

### Statistical Analysis

With the exception of screening experiments, all statistical significance was calculated in R (version 3.3.2). A Shapiro-Wilks normality test was used to determine whether data could be appropriately modeled by a Gaussian distribution. If data was non-Gaussian (Shapiro-Wilks p<0.05), a non-parametric Kruskal-Wallis test with Dunn's post-hoc analysis was used, and the false discovery rate was minimized using the Benjamini-Hochberg p-value adjustment. Otherwise, one-way ANOVA with Tukey post-hoc analysis was employed.

### Example 2: A Targeted Screen Identifies IL-3 and TNFα as Factors that Enhance ProT Cell Differentiation and Expansion

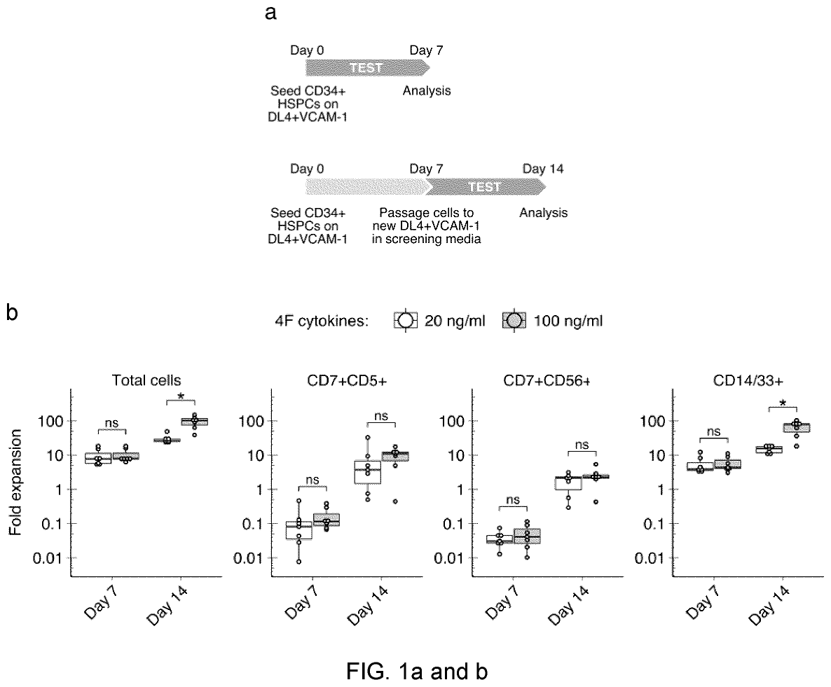

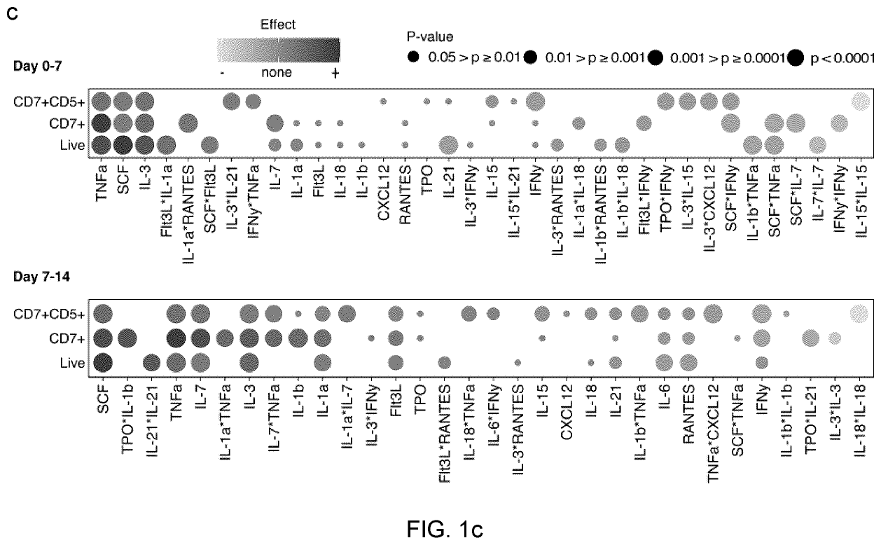

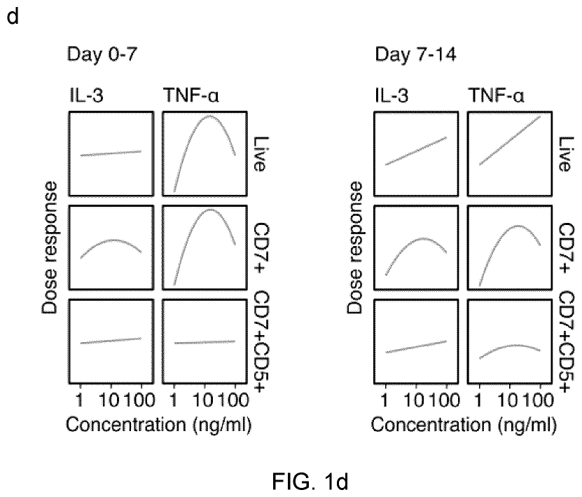

To identify factors that enhance proT cell differentiation and expansion, a two-part screen was conducted from day 0-7 and day 7-14 to separate the effects of screened molecules on HSPCs and differentiating proT cells (FIG. 1a). Screened molecules were tested at three doses each in combination using a definitive screening design (DSD) experiment and a linear regression model was fit to empirical data to estimate dose response.⁷ The response to factors was measured against a control condition that contained SCF, FLT3L, TPO and IL-7 cytokines as in Shukla et al.⁶ but using a lower concentration of 20 ng/ml each. This lower concentration was chosen so that SCF, FLT3L, TPO and IL-7 did not mask the effects of the screened factors. The decrease in concentration of SCF, FLT3L, TPO and IL-7 from 100 ng/ml to 20 ng/ml reduced the generation of CD14/33+ myeloid cells on day 14 without significantly affecting the number of CD7+CD5+ cells generated (FIG. 1b). Therefore, the lower concentration of 20 ng/ml did not significantly affect T lineage development.

The effect of the tested cytokines on total live cells, CD7+ lymphocytes, and CD7+CD5+ cells is shown in FIG. 1c. Medium gray indicates the effect of the control condition; dark gray to black indicates an effect greater than the control condition; and light gray to white indicates an effect lesser than the control condition. The size of each circle indicates the significance of the effect in the linear regression model. Combinations of factors indicate two-factor interactions (e.g., TPO*IL-1β) or quadratic effects (e.g., IL-21*IL-21) in the regression model. In addition to SCF and IL-7, the cytokines IL-3 and TNFα had string positive effects in both weeks. IL-3 and TNFα were selected for further investigation.

The dose response of cells to a range of concentrations of IL-3 and TNFα is shown in FIG. 1d. Each factor elicited a curved response in at least one population, indicating a concentration saturation point for that population. Working concentrations for the subsequent experiments were chosen as 10 ng/ml for IL-3 and 5 ng/ml for TNFα.

### Example 3: Analysis of HSPC proliferation in Response to IL-3 and TNFα

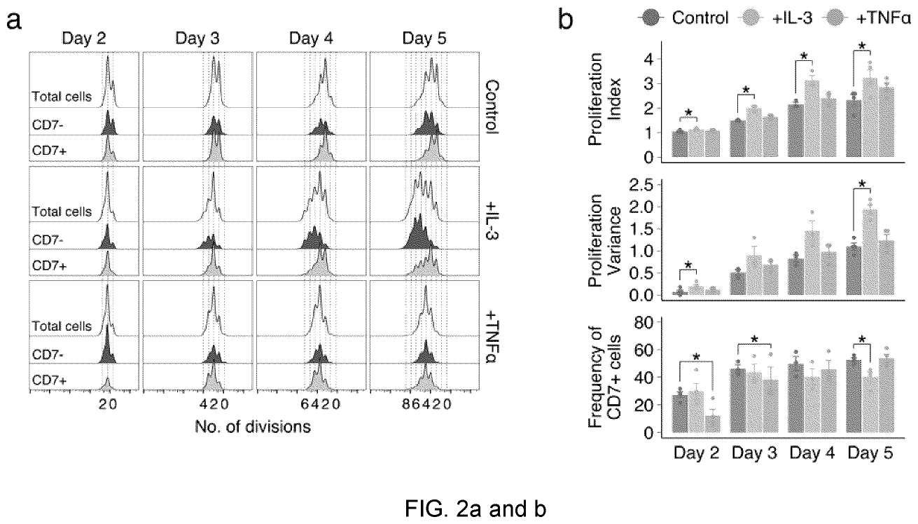

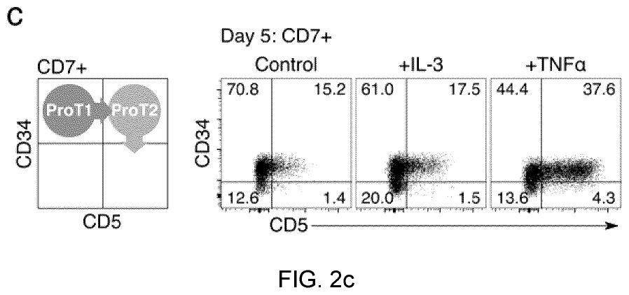

To track the number of cell divisions in response to each of IL-3 and TNFα, the HSPCs were first stained with carboxyfluorescein succinimidyl ester (CFSE). IL-3 or TNFα were added on day 0 and the number of divisions of each cell was analyzed on days 2, 3, 4 and 5. CD7− cells and CD7+ cells showed differential responses to each cytokine, with IL-3 predominantly affecting the CD7− fraction (FIG. 2a).

The proliferation statistics from CFSE data and the frequency of CD7+ cells on each day is shown in FIG. 2b. IL-3 treated cells underwent, on average, more divisions than the control group and had a significantly larger variance in the number of divisions than the control. TNFα treated cells were slow to upregulate CD7, while IL-3 treated cells upregulated CD7 comparably to the control but had a lower CD7+frequency than the control by day 5.

All treatment groups transitioned through a proT1 (CD34+CD7+CD5−) to a proT2 (CD34+CD7+CD5+) phenotype by day 5, as defined by Awong et al.¹ (FIG. 2c). The TNFα treatment group contained a higher proportion of proT2 cells. This finding was consistent with previous reports showing that TNFα accelerated the transition from DN1 to DN2 proT cells in mice.⁹ Given that T lineage development is driven by signaling through the Notch pathway, it was hypothesized that TNFα/Notch interactions may be responsible for the early T lineage induction.

### Example 4: Synergy Between TNFα and the Notch Pathway

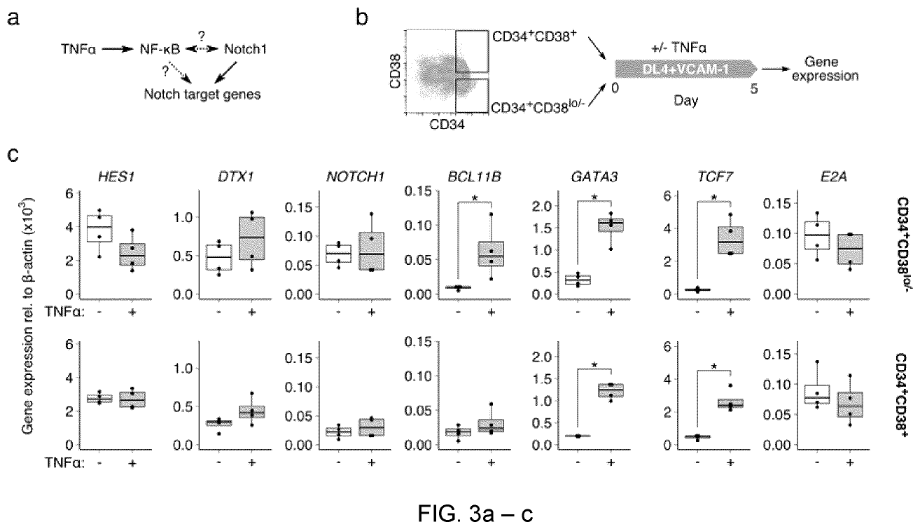

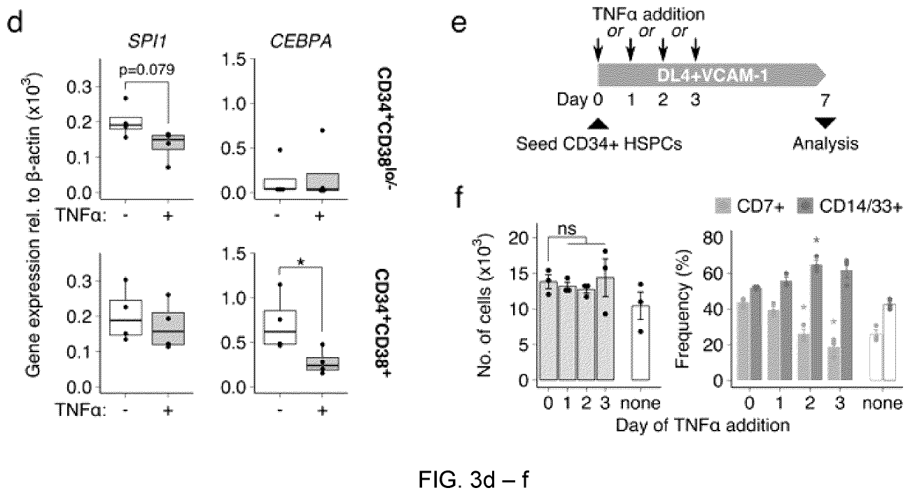

TNFα activates the NF-KB pathway, which may regulate Notch target genes or regulate Notch itself (FIG. 3a). To investigate if and how TNFα interacts with Notch, CD34+ HSPCs were first sorted into CD38lo/− and CD38+ fractions, and seeded separately on DL4+VCAM-1 with and without TNFα. Gene expression was measured using qPCR after 5 days of culture (FIG. 3b).

Both the CD38lo/− and CD38+ fractions upregulated GATA3 and TCF7 in response to TNFα, while only CD38lo/− HSPCs upregulated BCL11b, consistent with previous observations that CD34+CD38lo/− cells have a higher propensity for T lineage development.¹ No significant differences were observed in any other Notch target genes or NOTCH1 itself, indicating that TNFα was acting on target genes and not the Notch pathway itself (FIG. 3c). CD38lo/− but not CD38+ HSPCs downregulated SPI1 slightly in response to TNFα. In contrast, only the CD38+fraction significantly downregulated CEBPA when cultured with TNFα (FIG. 3d).

CD34+ HSPCs were placed on DL4+VCAM-1 for 7 days. TNFα was added on day 0, 1, 2, or 3 to elucidate any stage-specific effects on T lineage development (FIG. 3e). The total number and frequency of CD7+ and CD14/33+ cells on day 7 is shown in FIG. 3f. The late addition of TNFα to culture resulted in an increase in CD14/33+ myeloid lineage cells and a decrease in CD7+ lymphoid cells (FIG. 3f). Collectively, these results show that enhanced T lineage specification by TNFα is through regulation of select Notch target genes. In addition, this effect occurs early in development and TNFα has a positive effect on myeloid development once that process is initiated.

### Example 5: TNFα Compensates for Suboptimal Notch Activation

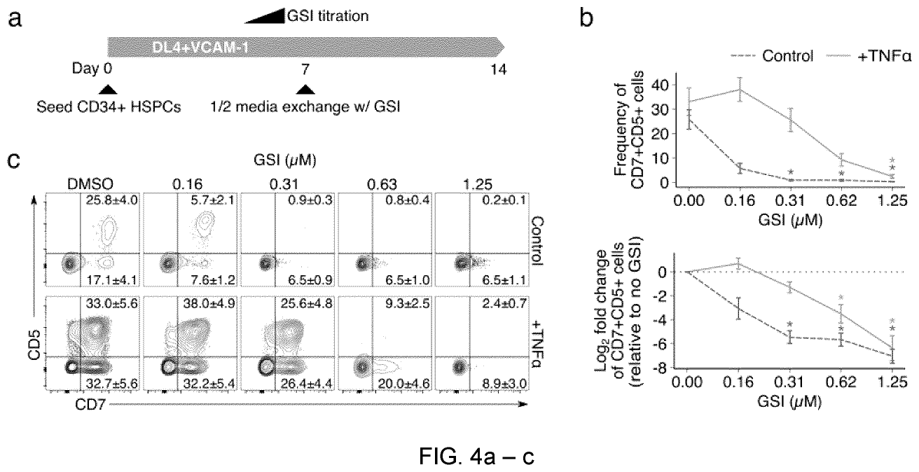

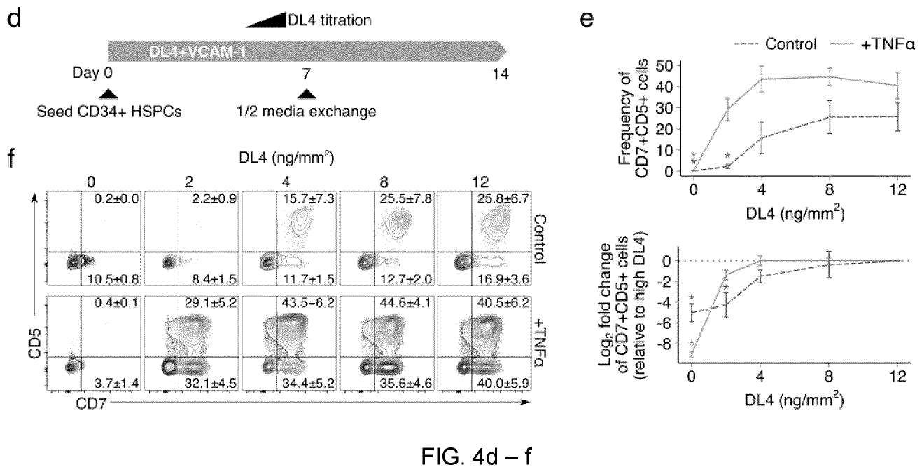

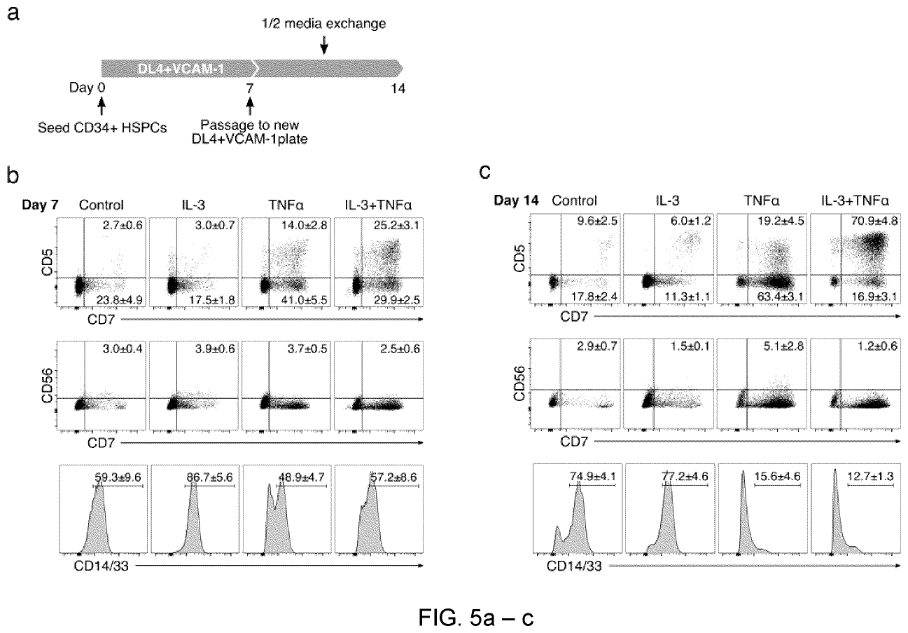

To validate the gene expression experiments shown in Example 4, the ability of TNFα to compensate for partial Notch pathway inhibition and to maintain T lineage development was investigated. To this end, CD34+ HSPCs were seeded on DL4+VCAM-1 for 14 days with increasing concentrations of γ-secretase inhibitor (GSI) to inhibit Notch activation (FIG. 4a). This experiment revealed that TNFα is capable of maintaining CD7+CD5+ proT cell generation with a higher concentration of GSI than the control condition (FIG. 4b). Representative flow cytometry plots showing the differential effects of Notch pathway inhibition in the presence or absence of TNFα are shown in FIG. 4c.

In parallel with the Notch pathway inhibition experiment described above, HSPCs were also seeded on DL4+VCAM-1 for 14 days with decreasing concentrations of DL4 (FIG. 4d). This experiment revealed that TNFα is capable of maintaining CD7+CD5+ proT cell development to the lowest coating concentration of DL4 tested (2 ng/mm²) (FIG. 4e). Representative flow cytometry plots showing differential proT cell induction with decreasing DL4 concentration in the presence or absence of TNFα are shown in FIG. 4f. Collectively, these results show that TNFα drives T lineage development even under conditions where the Notch pathway is partially inhibited or suboptimally activated.

### Example 6: IL-3 and TNFα Synergistically Enhance ProT Cell Expansion and Purity

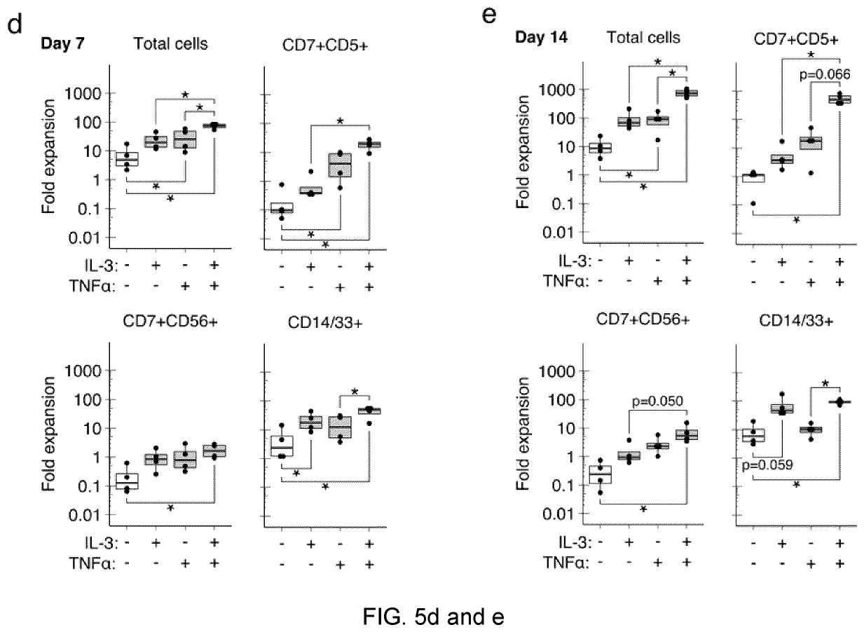

To assess the effects of combining IL-3 with TNFα on T lineage development, CD34+ HSPCs were placed on DL4+VCAM-1 and passaged to freshly coated plates every 7 days in the presence or absence of IL-3, TNFα, or both (IL-3+TNFα). Beginning in week two, a one-half media change was performed in between passages (FIG. 5a). Flow cytometry analysis of cells on day 7 and day 14 revealed an increase in the frequency of CD7+CD5+ proT cells treated with IL-3+TNFα compared to control conditions or IL-3 or TNFα alone (FIG. 5b and c). On day 14, approximately 70% of cells were CD7+CD5+ proT cells in the IL-3+TNFα group compared to less than 10% in the control group (FIG. 5c).

By day 7, the IL-3+TNFα treatment group contained significantly more cells than the control conditions or IL-3 or TNFα alone, and significantly more CD7+CD5+ proT cells than control or IL-3 groups (FIG. 5d).

By day 14, the number of cells in the IL-3+TNFα treatment group was approximately 100-fold greater than in the control group, and the number of CD7+CD5+ proT cells in the IL-3+TNFα treatment group was more than 500-fold greater than in the control group (FIG. 5e). The expansion of CD7+CD56+ NK cells and CD14/33+ myeloid cells was also greater in the IL-3+TNFα group. However, the NK and myeloid cell populations represented a small proportion of all cells, indicating that the combination of IL-3 and TNFα predominately expands T lineage cells.

### Example 7: TNFα Increases the Expression of IL-3 Receptor

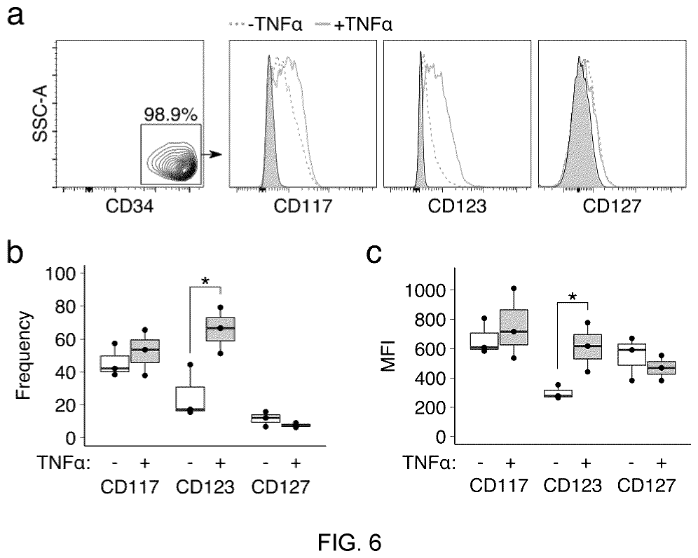

Next, the expression of CD117 (c-kit; SCF receptor), CD123 (IL-3 receptor) and CD127 (IL-7 receptor) on CD34+ HSPCs was evaluated in the presence or absence of TNFα stimulation for 24 hours. TNFα induced a significant increase in the frequency of CD123+ cells (FIGS. 6a and b). Additionally, the increased frequency of CD123+ cells was accompanied by an increase in the median fluorescent intensity (MFI) of CD123, indicating a higher cell surface receptor density following TNFα stimulation (FIG. 6c).

Together, these results provide a mechanism for the combined effect of IL-3 and TNFα: TNFα increases the overall responsiveness of the CD34+ HSPC population to IL-3 and couples the proliferative effect of IL-3 with the T lineage inducing effects of TNFα through regulation of Notch target genes.

### Example 8: The Combination of IL-3 and TNFα Compensates for the Absence of VCAM-1

To assess the requirement for VCAM-1 in T lineage development, CD34+ HSPCs were analyzed by flow cytometry after 14 days in the presence or absence of IL-3+TNFα, and further in the presence or absence of VCAM-1.

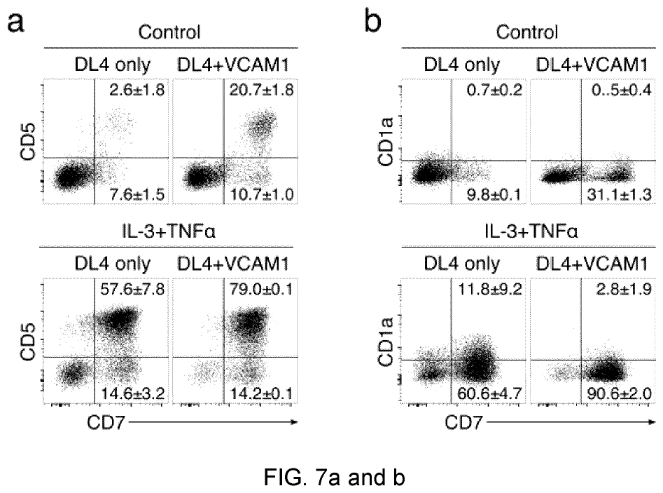

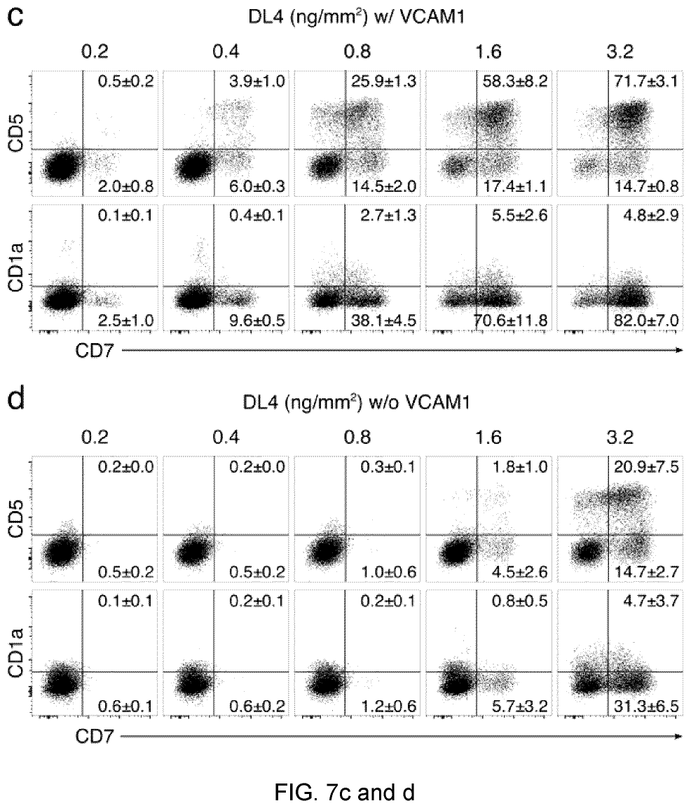

Removal of VCAM-1 from control conditions resulted in negligible CD7+CD5+ proT cell development, whereas IL-3+TNFα supported proT cell development in the absence of VCAM-1, although at a somewhat reduced frequency (FIG. 7a). Cells treated with IL-3+TNFα also had a higher proportion of CD7+CD1a+ preT cells than the control group, with the highest frequency found in conditions not containing VCAM-1 (FIG. 7b). This finding is consistent with a previous report showing that human T lineage commitment (i.e., transition from proT cells to preT cells) requires a decrease in Notch pathway activity.¹⁰

Following up on the finding that TNFα supported T lineage development with reduced concentrations of DL4 (FIG. 4d-f), a similar titration experiment was carried out at an even lower range of DL4 concentrations in the presence of IL-3+TNFα. Coating concentrations as low as 0.8 ng/mm² of DL4 supported proT and preT cell development (FIG. 7c). This DL4 coating concentration represents a 30-fold reduction when compared to the concentration reported by Shukla et al.⁶

The DL4 titration experiment was repeated in the absence of VCAM1. This experiment revealed that a coating concentration of 3.2 ng/mm² of DL4, or 7.5-fold less than Shukla et al.⁶, supported proT and preT cell development in the absence of VCAM-1 (FIG. 7d). These results demonstrate that the combination of IL-3 and TNFα can compensate for the absence of VCAM-1 in T lineage development and T lineage commitment.

### Example 9: TNFα Compensates for the Absence of TPO

To assess the requirement for TPO in T lineage development, CD34+ HSPCs were analyzed by flow cytometry after 14 days in the presence or absence of TNFα, and further in the presence or absence of TPO.

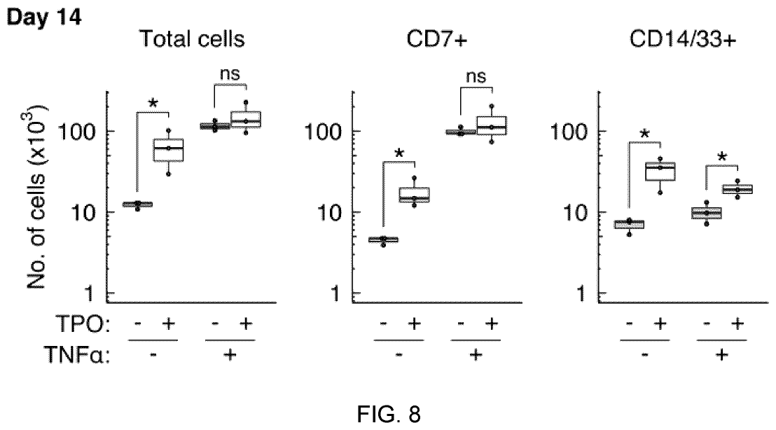

The removal of TPO was not detrimental to CD7+ lymphoid cell development when TNFα was present, but the number of CD7+ cells decreased significantly in the absence of TNFα (FIG. 8). Furthermore, the removal of TPO decreased the number of CD14/33+ myeloid cells with or without TNFα (FIG. 8). Together, these results show that TNFα supports T lineage development in the absence of TPO.

### Example 10: Summary of the Non-Limiting Exemplary Embodiments

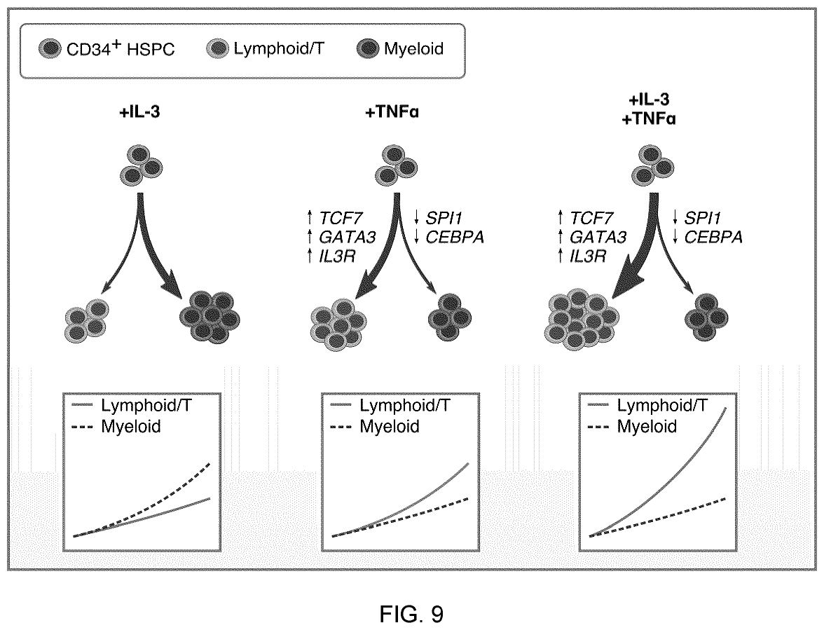

As illustrated in FIG. 9, IL-3 induces cell proliferation but, since the IL-3 receptor (CD123) is not widely expressed on CD34+ HSPCs, this effect is limited to cells that differentiate towards a non-lymphoid (CD7-) lineage. On its own, TNFα induces proliferation and T-lineage differentiation, in part through the upregulation of Notch target genes such as TCF7 and GATA3, and consequent downregulation of pro-myeloid lineage genes SPI1 and CEBPA. This effect occurs early to initiate the T cell developmental program, with delayed addition of TNFα having a positive effect on CD14/33+ myeloid cell expansion once a myeloid lineage program has been initiated (see FIGS. 3e and f).

Upregulation of the IL-3 receptor by TNFα in CD34+ HSPCs makes these cells responsive to IL-3, whereas only a small subset of this population is IL-3 responsive in the absence of TNFα. This couples the proliferative effect of IL-3 with the T lineage inducing effect of TNFα. The number of CD14/33+ myeloid lineage cells in IL-3 and IL-3+TNFα conditions was similar (see FIGS. 5d and e). This finding supports a model where TNFα does not directly suppress myeloid development. The downregulation of SPI1 and CEBPA by TNFα is likely a consequence of enhanced BCL11 b expression—a negative regulator of non-T development—in those cells that have initiated T lineage development, as opposed to a population-wide decrease in pro-myeloid gene expression. However, the proliferative capacity of proT cells stimulated by IL-3 and TNFα greatly exceeded that of the myeloid population, leading to significantly enriched populations of T lineage cells, including proT cells and derivatives of proT cells, such as preT cells and mature T cells.

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

### DOCUMENTS CITED

1. Awong G, Herer E, Surh C D, Dick J E, La Motte-Mohs R N, Zúñiga-Pflücker J C. Characterization in vitro and engraftment potential in vivo of human progenitor T cells generated from hematopoietic stem cells. Blood. 2009; 114 (5):972-982. doi:10.1182/blood-2008-10-187013.
2. La Motte-Mohs R N, Herer E, Zúñiga-Pflücker J C. Induction of T-cell development from human cord blood hematopoietic stem cells by Delta-like 1 in vitro. Blood. 2005; 105 (4):1431-1439. doi:10.1182/blood-2004-04-1293.
3. Ikawa T, Hirose S, Masuda K, et al. An essential developmental checkpoint for production of the T cell lineage. Science. 2010; 329 (5987):93-96. doi:10.1126/science.1188995.
4. Taqvi S, Dixit L, Roy K. Biomaterial-based notch signaling for the differentiation of hematopoietic stem cells into T cells. J Biomed Mater Res—Part A. 2006; 79 (3):689-697. doi:10.1002/jbm.a.30916.
5. Roccio M, Gobaa S, Lutolf M P. High-throughput clonal analysis of neural stem cells in microarrayed artificial niches. Integr Biol. 2012; 4 (4):391. doi:10.1039/c2ib00070a.
6. Shukla S et al. Progenitor T-cell differentiation from hematopoietic stem cells using Delta-like-4 and VCAM-1. Nat. Methods. 2017; 14 (5):531-538. doi:10.1038/nmeth.4258.
7. Jones B and Nachtsheim CJ. A class of three-level designs for definitive screening in the presence of second-order effects. Journal of Quality Technology. 2011; 43 (1):1-15.
8. Jones B and Nachtsheim C J. Blocking Schemes for Definitive Screening Designs. Technometrics. 2016; 58:74-83. doi:10.1080/00401706.2015.1013777.
9. Zúñiga-Pflücker J C, Di J, Lenardo M J. Requirement for TNF-alpha and IL-1 alpha in fetal thymocyte commitment and differentiation. Science. 1995; 268 (5219):1906-1909. doi: 10.1126/science.7541554.
10. Van de Walle I et al. An Early Decrease in Notch Activation Is Required for Human TCR-Lineage Differentiation at the Expense of TCR-T Cells. Blood. 2009:113 (13):2988-2998.

doi:10.1182/blood-2008-06-164871.

What is claimed is:

1. A serum-free culture medium comprising tumor necrosis factor alpha (TNFα), interleukin 3 (IL-3) and a Notch ligand, or functionally equivalent fragments thereof, wherein the Notch ligand or fragment thereof is adsorbed or immobilized to a substrate.

2. The serum-free culture medium of claim 1, further comprising at least one of: stem cell factor (SCF), interleukin 7 (IL-7) or FMS-like tyrosine kinase 3 ligand (FLT3L), or functionally equivalent fragments thereof.

3. The serum-free culture medium of claim 1, further comprising at least two of: stem cell factor (SCF), interleukin 7 (IL-7) or FMS-like tyrosine kinase 3 ligand (FLT3L), or functionally equivalent fragments thereof.

4. The serum-free culture medium of claim 1, further comprising stem cell factor (SCF), interleukin 7 (IL-7) and FMS-like tyrosine kinase 3 ligand (FLT3L), or functionally equivalent fragments thereof.

5. The serum-free culture medium of any one of claims 1-4, further comprising an adhesion ligand and/or an integrin ligand, or a functionally equivalent fragment thereof, adsorbed or immobilized to a substrate.

6. The serum-free culture medium of any one of claims 1-5, further comprising thrombopoietin (TPO) or a functionally equivalent fragment thereof.

7. The serum-free culture medium of any one of claims 1-6, wherein the TNFα is present at a concentration of between about 1 ng/ml and about 50 ng/ml.

8. The serum-free culture medium of any one of claims 1-7, wherein the IL-3 is present at a concentration of between about 1 ng/ml and about 50 ng/ml.

9. The serum-free culture medium of any one of claims 2-8, wherein the SCF, IL-7 and FLT3L are each present at a concentration of between about 5 ng/ml and about 200 ng/ml.

10. The serum-free culture medium of any one of claims 1-9, wherein the Notch ligand is Delta-like 4 (DL4), Delta-like 1 (DL1), Jagged 2 (JAG2), or a combination thereof.

11. The serum-free culture medium of any one of claims 1-10, wherein the substrate comprises one or more beads contained within the medium.

12. The serum-free culture medium of any one of claims 1-11, wherein the Notch ligand is adsorbed or immobilized to the substrate at a coating concentration of between about 0.5 ng/mm2 and about 50 ng/mm2.

13. The serum-free culture medium of any one of claims 1-12, further comprising one or more of bovine serum albumin, insulin, transferrin, low-density lipoprotein, ascorbic acid, 2-mercaptoethanol, penicillin and streptomycin.

14. A kit comprising the serum-free culture medium of any one of claims 1-13 and at least one container.

15. A kit comprising:
a. a serum-free culture medium comprising TNFα, IL-3, or functionally equivalent fragments thereof; and
b. a coating medium comprising a Notch ligand or a functionally equivalent fragment thereof.

16. The kit of claim 15, wherein the serum-free culture medium further comprises at least one of: SCF, IL-7 or FLT3L, or functionally equivalent fragments thereof.

17. The kit of claim 15 or 16, wherein the serum-free culture medium further comprises TPO or a functionally equivalent fragment thereof.

18. The kit of any one of claims 15-17, wherein the coating medium further comprises an adhesion ligand and/or an integrin ligand, or a functionally equivalent fragment thereof.

19. The kit of any one of claims 15-18, wherein the Notch ligand is DL4, DL1, JAG2, or a combination thereof.

20. The kit of any one of claims 15-19, further comprising one or more cell culture plates or dishes.

21. A method of generating a progenitor T cell or a derivative thereof, the method comprising culturing stem and/or progenitor cells in a serum-free medium in the presence of TNFα, IL-3 and a Notch ligand, or functionally equivalent fragments thereof, wherein the Notch ligand is adsorbed or immobilized to a substrate.

22. The method of claim 21, wherein the derivative is a preT cell, immature T cell or mature T cell.

23. The method of claim 21, further comprising differentiating the progenitor T cell to a preT cell, immature T cell or a mature T cell.

24. A method of generating natural killer (NK) cells, the method comprising culturing stem and/or progenitor cells in a serum-free medium in the presence of TNFα, IL-3 and a Notch ligand, or functionally equivalent fragments thereof, wherein the Notch ligand is adsorbed or immobilized to a substrate.

25. The method of any one of claims 21-24, wherein the culturing is further in the presence of at least one of: SCF, IL-7 or FLT3L, or functionally equivalent fragments thereof.

26. The method of any one of claims 21-25, wherein the TNFα and the IL-3 are provided to the cells at the same time.

27. The method of any one of claims 21-25, wherein the TNFα is provided to the cells prior to the IL-3.

28. The method of any one of claims 21-25 and 27, wherein the TNFα is provided to the cells 1, 2, 3, 4 or 5 days prior to the IL-3.

29. The method of any one of claims 21-28, wherein the culturing is further in the presence of an adhesion ligand and/or an integrin ligand, or a functionally equivalent fragment thereof, adsorbed or immobilized to a substrate.

30. The method of any one of claims 21-29, wherein the culturing is further in the presence of TPO or a functionally equivalent fragment thereof.

31. The method of any one of claims 21-30, wherein the TNFα is provided at a concentration of between about 1 ng/ml and about 50 ng/ml in the culture.

32. The method of any one of claims 21-31, wherein the IL-3 is provided at a concentration of between about 1 ng/ml and about 50 ng/ml in the culture.

33. The method of any one of claims 25-32, wherein the SCF, IL-7 and FLT3L are each provided at a concentration of between about 5 ng/ml and about 200 ng/ml in the culture.

34. The method of any one of claims 21-33, wherein the Notch ligand is DL4, DL1, JAG2, or a combination thereof.

35. The method of any one of claims 21-34, wherein the Notch ligand is adsorbed or immobilized to the substrate at a coating concentration of between about 0.5 ng/mm2 and about 50 ng/mm2.

36. The method of any one of claims 21-35, wherein the substrate comprises a surface of a cell culture plate or dish, or a culture bag.

37. The method of any one of claims 21-35, wherein the substrate comprises one or more beads contained within the medium.

38. The method of any one of claims 21-37, wherein the stem and/or progenitor cells are human cells.

39. The method of any one of claims 21-38, wherein the stem and/or progenitor cells are CD34+ hematopoietic stem and progenitor cells (HSPCs).

40. The method of claim 39, wherein the CD34+ HSPCs are derived from umbilical cord blood, peripheral blood, bone marrow, embryonic stem cells or induced pluripotent stem cells.

41. The method of any one of claims 21-23 and 25-40, wherein the progenitor T cells comprise CD7+ cells, CD7+CD5+ cells, CD7+CD5+CD34+, CD7+CD5+CD45RA+ cell and/or CD7+CD5+CD1a+ cell.

42. The method of any one of claims 24-40, wherein the NK cells comprise CD7+CD56+ cells.

43. The method of any one of claims 21-23 and 25-41, wherein the generated progenitor T cells have a higher cell surface density of IL-3 receptors compared to progenitor T cells generated by culturing in a medium without TNFα.

44. The method of any one of claims 21-23, 25-41 and 43, wherein the culturing further comprises generating progenitor T cells and, optionally, derivatives of the generated progenitor T cells.

45. The method of claim 44, wherein the derivatives of the generated progenitor T cells comprise CD4+CD8+ cells, CD4+CD3+ cells and/or CD8+CD3+ cells.

46. The method of any one of claims 24-40 and 42, wherein the culturing further comprises generating NK cells and, optionally, derivatives of the generated NK cells.

47. The method of any one of claims 21-46, wherein the culturing is for a time of at least 7 days or at least 14 days.

48. A progenitor T cell or a derivative of a progenitor T cell generated by the method of any one of claims 21-23, 25-41, 43-45 and 47.

49. A population of progenitor T cells made by the method according to any one of claims 21-23, 25-41, 43-45 and 47.

50. An NK cell or a derivative of an NK cell generated by the method of any one of claims 24-40, 42 and 46-47.

51. A method for enhancing the immune response in a subject, the method comprising administering to the subject an effective number of the progenitor T cells or derivatives thereof of claim 48.

52. A method for increasing the number of T cells in a subject, the method comprising administering to the subject an effective number of the progenitor T cells or derivatives thereof of claim 48.

53. A method for enhancing the immune response in a subject, the method comprising administering to the subject an effective number of the NK cells or derivatives thereof of claim 50.

54. A method for increasing the number of NK cells in a subject, the method comprising administering to the subject an effective number of the NK cells or derivatives thereof of claim 50.

55. The method of claim 51 or 52, wherein the administered progenitor T cells or derivatives thereof are autologous.

56. The method of claim 51 or 52, wherein the administered progenitor T cells or derivatives thereof are allogeneic.

57. The method of claim 53 or 54, wherein the administered NK cells or derivatives thereof are autologous.

58. The method of claim 53 or 54, wherein the administered NK cells or derivatives thereof are allogeneic.

59. The method of any one of claims 51-58, wherein the subject has or is at risk of having an immune deficiency.

60. The method of claim 59, wherein the immune deficiency is a T cell deficiency.

61. The method of claim 59, wherein the immune deficiency is an NK cell deficiency.

62. The method of any one of claims 59-61, wherein the immune deficiency is caused by a medical condition, a chemical exposure and/or a radiation exposure.

63. The method of claim 62, wherein the medical condition is a cancer, a bone marrow failure, an anemia, a primary immunodeficiency disorder, an autoimmune disease, a partial thymectomy, an organ transplant, a viral infection, a bacterial infection, a fungal infection and/or idiopathic CD4+ T-lymphocytopenia.

64. The method of claim 63, wherein the viral infection is an HIV infection.

65. The method of claim 62, wherein the chemical exposure comprises chemotherapy, anti-inflammatory drug therapy, workplace-related chemical exposure or accidental poisoning with a chemical substance.

66. The method of claim 62, wherein the radiation exposure comprises radiotherapy, workplace-related radioisotope exposure, accidental poisoning with a radioisotope, nuclear meltdown or nuclear fallout.

67. The method of any one of claims 51-66, wherein the subject is a human.

68. A method of enhancing an immune response in a patient in need thereof by producing a population of progenitor T cells or derivatives thereof according to the method of any one of claims 21-23, 25-41, 43-45 and 47 and administering the population to the patient.
